SEQUENCE LISTING (29 sequences)

SEQ ID NO: 1 - protein, 1025 aa, Homo sapiens
  MISC_FEATURE (86)..(86): Xaa= Pro ou Ser
MEPFTNDRLQLPRNMIENSMFEEEPDVVDLAKEPCLHPLEPDEVEYEPRGSRLLVRGLGEHEMEEDEEDYESSAKLLGMSFMNRSXGLRNSATGYRQSPDGACSVPSARTMVVCAFVIVVAVSVIMVIYLLPRCTFTKEGCHKKNQSIGLIQPFATNGKLFPWAQIRLPTAVVPLRYELSLHPNLTSMTFRGSVTISVQALQVTWNIILHSTGHNISRVTFMSAVSSQEKQAEILEYAYHGQIAIVAPEALLAGHNYTLKIEYSANISSSYYGFYGFSYTDESNEKKYFAATQFEPLAARSAFPCFDEPAFKATFIIKIIRDEQYTALSNMPKKSSVVLDDGLVQDEFSESVKMSTYLVAFIVGEMKNLSQDVNGTLVSIYAVPEKIGQVHYALETTVKLLEFFQNYFEIQYPLKKLDLVAIPDFEAGAMENWGLLTFREETLLYDSNTSSMADRKLVTKIIAHELAHQWFGNLVTMKWWNDLWLNEGFATFMEYFSLEKIFKELSSYEDFLDARFKTMKKDSLNSSHPISSSVQSSEQIEEMFDSLSYFKGSSLLLMLKTYLSEDVFQHAVVLYLHNHSYASIQSDDLWDSFNEVTNQTLDVKRMMKTWTLQKGFPLVTVQKKGKELFIQQERFFLNMKPEIQPSDTSYLWHIPLSYVTEGRNYSKYQSVSLLDKKSGVINLTEEVLWVKVNINMNGYYIVHYADDDWEALIHQLKINPYVLSDKDRANLINNIFELAGLGKVPLKRAFDLINYLGNENHTXPITEALFQTDLIYNLLEKLGYMDLASRLVTRVFKLLQNQIQQQTWTDEGTPSMRELRSALLEFACTHNLGNCSTTAMKLFDDWMASNGTQSLPTDVMTTVFKVGAKTDKGWSFLLGKYISIGSEAEKNKILEALASSEDVRKLYWLMKSSLNGDNFRTQKLSFIIRTVGRHFPGHLLAWDFVKENWNKLVQKFPLGSYTXQNIVAGSTYLFSTKTHLSEVQAFFENQSEATFRLRCVQEALEVIQLNIQWMEKNLKSLTWWL

SEQ ID NO: 2 - protein, 1011 aa, Homo sapiens
  MISC_FEATURE (72)..(72): Xaa= Pro ou Ser
MIENSMFEEEPDVVDLAKEPCLHPLEPDEVEYEPRGSRLLVRGLGEHEMEEDEEDYESSAKLLGMSFMNRSXGLRNSATGYRQSPDGACSVPSARTMVVCAFVIVVAVSVIMVIYLLPRCTFTKEGCHKKNQSIGLIQPFATNGKLFPWAQIRLPTAVVPLRYELSLHPNLTSMTFRGSVTISVQALQVTWNIILHSTGHNISRVTFMSAVSSQEKQAEILEYAYHGQIAIVAPEALLAGHNYTLKIEYSANISSSYYGFYGFSYTDESNEKKYFAATQFEPLAARSAFPCFDEPAFKATFIIKIIRDEQYTALSNMPKKSSVVLDDGLVQDEFSESVKMSTYLVAFIVGEMKNLSQDVNGTLVSIYAVPEKIGQVHYALETTVKLLEFFQNYFEIQYPLKKLDLVAIPDFEAGAMENWGLLTFREETLLYDSNTSSMADRKLVTKIIAHELAHQWFGNLVTMKWWNDLWLNEGFATFMEYFSLEKIFKELSSYEDFLDARFKTMKKDSLNSSHPISSSVQSSEQIEEMFDSLSYFKGSSLLLMLKTYLSEDVFQHAVVLYLHNHSYASIQSDDLWDSFNEVTNQTLDVKRMMKTWTLQKGFPLVTVQKKGKELFIQQERFFLNMKPEIQPSDTSYLWHIPLSYVTEGRNYSKYQSVSLLDKKSGVINLTEEVLWVKVNINMNGYYIVHYADDDWEALIHQLKINPYVLSDKDRANLINNIFELAGLGKVPLKRAFDLINYLGNENHTXPITEALFQTDLIYNLLEKLGYMDLASRLVTRVFKLLQNQIQQQTWTDEGTPSMRELRSALLEFACTHNLGNCSTTAMKLFDDWMASNGTQSLPTDVMTTVFKVGAKTDKGWSFLLGKYISIGSEAEKNKILEALASSEDVRKLYWLMKSSLNGDNFRTQKLSFIIRTVGRHFPGHLLAWDFVKENWNKLVQKFPLGSYTXQNIVAGSTYLFSTKTHLSEVQAFFENQSEATFRLRCVQEALEVIQLNIQWMEKNLKSLTWWL

SEQ ID NO: 3 - protein, 1006 aa, Homo sapiens
  MISC_FEATURE (67)..(67): Xaa= Pro ou Ser
MFEEEPDVVDLAKEPCLHPLEPDEVEYEPRGSRLLVRGLGEHEMEEDEEDYESSAKLLGMSFMNRSXGLRNSATGYRQSPDGACSVPSARTMVVCAFVIVVAVSVIMVIYLLPRCTFTKEGCHKKNQSIGLIQPFATNGKLFPWAQIRLPTAVVPLRYELSLHPNLTSMTFRGSVTISVQALQVTWNIILHSTGHNISRVTFMSAVSSQEKQAEILEYAYHGQIAIVAPEALLAGHNYTLKIEYSANISSSYYGFYGFSYTDESNEKKYFAATQFEPLAARSAFPCFDEPAFKATFIIKIIRDEQYTALSNMPKKSSVVLDDGLVQDEFSESVKMSTYLVAFIVGEMKNLSQDVNGTLVSIYAVPEKIGQVHYALETTVKLLEFFQNYFEIQYPLKKLDLVAIPDFEAGAMENWGLLTFREETLLYDSNTSSMADRKLVTKIIAHELAHQWFGNLVTMKWWNDLWLNEGFATFMEYFSLEKIFKELSSYEDFLDARFKTMKKDSLNSSHPISSSVQSSEQIEEMFDSLSYFKGSSLLLMLKTYLSEDVFQHAVVLYLHNHSYASIQSDDLWDSFNEVTNQTLDVKRMMKTWTLQKGFPLVTVQKKGKELFIQQERFFLNMKPEIQPSDTSYLWHIPLSYVTEGRNYSKYQSVSLLDKKSGVINLTEEVLWVKVNINMNGYYIVHYADDDWEALIHQLKINPYVLSDKDRANLINNIFELAGLGKVPLKRAFDLINYLGNENHTXPITEALFQTDLIYNLLEKLGYMDLASRLVTRVFKLLQNQIQQQTWTDEGTPSMRELRSALLEFACTHNLGNCSTTAMKLFDDWMASNGTQSLPTDVMTTVFKVGAKTDKGWSFLLGKYISIGSEAEKNKILEALASSEDVRKLYWLMKSSLNGDNFRTQKLSFIIRTVGRHFPGHLLAWDFVKENWNKLVQKFPLGSYTXQNIVAGSTYLFSTKTHLSEVQAFFENQSEATFRLRCVQEALEVIQLNIQWMEKNLKSLTWWL

SEQ ID NO: 4 - protein, 1025 aa, Homo sapiens
MEPFTNDRLQLPRNMIENSMFEEEPDVVDLAKEPCLHPLEPDEVEYEPRGSRLLVRGLGEHEMEEDEEDYESSAKLLGMSFMNRSSGLRNSATGYRQSPDGACSVPSARTMVVCAFVIVVAVSVIMVIYLLPRCTFTKEGCHKKNQSIGLIQPFATNGKLFPWAQIRLPTAVVPLRYELSLHPNLTSMTFRGSVTISVQALQVTWNIILHSTGHNISRVTFMSAVSSQEKQAEILEYAYHGQIAIVAPEALLAGHNYTLKIEYSANISSSYYGFYGFSYTDESNEKKYFAATQFEPLAARSAFPCFDEPAFKATFIIKIIRDEQYTALSNMPKKSSVVLDDGLVQDEFSESVKMSTYLVAFIVGEMKNLSQDVNGTLVSIYAVPEKIGQVHYALETTVKLLEFFQNYFEIQYPLKKLDLVAIPDFEAGAMENWGLLTFREETLLYDSNTSSMADRKLVTKIIAHELAHQWFGNLVTMKWWNDLWLNEGFATFMEYFSLEKIFKELSSYEDFLDARFKTMKKDSLNSSHPISSSVQSSEQIEEMFDSLSYFKGSSLLLMLKTYLSEDVFQHAVVLYLHNHSYASIQSDDLWDSFNEVTNQTLDVKRMMKTWTLQKGFPLVTVQKKGKELFIQQERFFLNMKPEIQPSDTSYLWHIPLSYVTEGRNYSKYQSVSLLDKKSGVINLTEEVLWVKVNINMNGYYIVHYADDDWEALIHQLKINPYVLSDKDRANLINNIFELAGLGKVPLKRAFDLINYLGNENHTAPITEALFQTDLIYNLLEKLGYMDLASRLVTRVFKLLQNQIQQQTWTDEGTPSMRELRSALLEFACTHNLGNCSTTAMKLFDDWMASNGTQSLPTDVMTTVFKVGAKTDKGWSFLLGKYISIGSEAEKNKILEALASSEDVRKLYWLMKSSLNGDNFRTQKLSFIIRTVGRHFPGHLLAWDFVKENWNKLVQKFPLGSYTIQNIVAGSTYLFSTKTHLSEVQAFFENQSEATFRLRCVQEALEVIQLNIQWMEKNLKSLTWWL

SEQ ID NO: 5 - protein, 1025 aa, Homo sapiens
MEPFTNDRLQLPRNMIENSMFEEEPDVVDLAKEPCLHPLEPDEVEYEPRGSRLLVRGLGEHEMEEDEEDYESSAKLLGMSFMNRSPGLRNSATGYRQSPDGACSVPSARTMVVCAFVIVVAVSVIMVIYLLPRCTFTKEGCHKKNQSIGLIQPFATNGKLFPWAQIRLPTAVVPLRYELSLHPNLTSMTFRGSVTISVQALQVTWNIILHSTGHNISRVTFMSAVSSQEKQAEILEYAYHGQIAIVAPEALLAGHNYTLKIEYSANISSSYYGFYGFSYTDESNEKKYFAATQFEPLAARSAFPCFDEPAFKATFIIKIIRDEQYTALSNMPKKSSVVLDDGLVQDEFSESVKMSTYLVAFIVGEMKNLSQDVNGTLVSIYAVPEKIGQVHYALETTVKLLEFFQNYFEIQYPLKKLDLVAIPDFEAGAMENWGLLTFREETLLYDSNTSSMADRKLVTKIIAHELAHQWFGNLVTMKWWNDLWLNEGFATFMEYFSLEKIFKELSSYEDFLDARFKTMKKDSLNSSHPISSSVQSSEQIEEMFDSLSYFKGSSLLLMLKTYLSEDVFQHAVVLYLHNHSYASIQSDDLWDSFNEVTNQTLDVKRMMKTWTLQKGFPLVTVQKKGKELFIQQERFFLNMKPEIQPSDTSYLWHIPLSYVTEGRNYSKYQSVSLLDKKSGVINLTEEVLWVKVNINMNGYYIVHYADDDWEALIHQLKINPYVLSDKDRANLINNIFELAGLGKVPLKRAFDLINYLGNENHTAPITEALFQTDLIYNLLEKLGYMDLASRLVTRVFKLLQNQIQQQTWTDEGTPSMRELRSALLEFACTHNLGNCSTTAMKLFDDWMASNGTQSLPTDVMTTVFKVGAKTDKGWSFLLGKYISIGSEAEKNKILEALASSEDVRKLYWLMKSSLNGDNFRTQKLSFIIRTVGRHFPGHLLAWDFVKENWNKLVQKFPLGSYTIQNIVAGSTYLFSTKTHLSEVQAFFENQSEATFRLRCVQEALEVIQLNIQWMEKNLKSLTWWL

SEQ ID NO: 6 - protein, 1025 aa, Homo sapiens [sequence omitted]

SEQ ID NO: 7 - protein, 1025 aa, Homo sapiens [sequence omitted]

SEQ ID NO: 8 - protein, 1011 aa, Homo sapiens
MIENSMFEEEPDVVDLAKEPCLHPLEPDEVEYEPRGSRLLVRGLGEHEMEEDEEDYESSAKLLGMSFMNRSSGLRNSATGYRQSPDGACSVPSARTMVVCAFVIVVAVSVIMVIYLLPRCTFTKEGCHKKNQSIGLIQPFATNGKLFPWAQIRLPTAVVPLRYELSLHPNLTSMTFRGSVTISVQALQVTWNIILHSTGHNISRVTFMSAVSSQEKQAEILEYAYHGQIAIVAPEALLAGHNYTLKIEYSANISSSYYGFYGFSYTDESNEKKYFAATQFEPLAARSAFPCFDEPAFKATFIIKIIRDEQYTALSNMPKKSSVVLDDGLVQDEFSESVKMSTYLVAFIVGEMKNLSQDVNGTLVSIYAVPEKIGQVHYALETTVKLLEFFQNYFEIQYPLKKLDLVAIPDFEAGAMENWGLLTFREETLLYDSNTSSMADRKLVTKIIAHELAHQWFGNLVTMKWWNDLWLNEGFATFMEYFSLEKIFKELSSYEDFLDARFKTMKKDSLNSSHPISSSVQSSEQIEEMFDSLSYFKGSSLLLMLKTYLSEDVFQHAVVLYLHNHSYASIQSDDLWDSFNEVTNQTLDVKRMMKTWTLQKGFPLVTVQKKGKELFIQQERFFLNMKPEIQPSDTSYLWHIPLSYVTEGRNYSKYQSVSLLDKKSGVINLTEEVLWVKVNINMNGYYIVHYADDDWEALIHQLKINPYVLSDKDRANLINNIFELAGLGKVPLKRAFDLINYLGNENHTAPITEALFQTDLIYNLLEKLGYMDLASRLVTRVFKLLQNQIQQQTWTDEGTPSMRELRSALLEFACTHNLGNCSTTAMKLFDDWMASNGTQSLPTDVMTTVFKVGAKTDKGWSFLLGKYISIGSEAEKNKILEALASSEDVRKLYWLMKSSLNGDNFRTQKLSFIIRTVGRHFPGHLLAWDFVKENWNKLVQKFPLGSYTIQNIVAGSTYLFSTKTHLSEVQAFFENQSEATFRLRCVQEALEVIQLNIQWMEKNLKSLTWWL

SEQ ID NO: 9 - protein, 1011 aa, Homo sapiens
MIENSMFEEEPDVVDLAKEPCLHPLEPDEVEYEPRGSRLLVRGLGEHEMEEDEEDYESSAKLLGMSFMNRSPGLRNSATGYRQSPDGACSVPSARTMVVCAFVIVVAVSVIMVIYLLPRCTFTKEGCHKKNQSIGLIQPFATNGKLFPWAQIRLPTAVVPLRYELSLHPNLTSMTFRGSVTISVQALQVTWNIILHSTGHNISRVTFMSAVSSQEKQAEILEYAYHGQIAIVAPEALLAGHNYTLKIEYSANISSSYYGFYGFSYTDESNEKKYFAATQFEPLAARSAFPCFDEPAFKATFIIKIIRDEQYTALSNMPKKSSVVLDDGLVQDEFSESVKMSTYLVAFIVGEMKNLSQDVNGTLVSIYAVPEKIGQVHYALETTVKLLEFFQNYFEIQYPLKKLDLVAIPDFEAGAMENWGLLTFREETLLYDSNTSSMADRKLVTKIIAHELAHQWFGNLVTMKWWNDLWLNEGFATFMEYFSLEKIFKELSSYEDFLDARFKTMKKDSLNSSHPISSSVQSSEQIEEMFDSLSYFKGSSLLLMLKTYLSEDVFQHAVVLYLHNHSYASIQSDDLWDSFNEVTNQTLDVKRMMKTWTLQKGFPLVTVQKKGKELFIQQERFFLNMKPEIQPSDTSYLWHIPLSYVTEGRNYSKYQSVSLLDKKSGVINLTEEVLWVKVNINMNGYYIVHYADDDWEALIHQLKINPYVLSDKDRANLINNIFELAGLGKVPLKRAFDLINYLGNENHTAPITEALFQTDLIYNLLEKLGYMDLASRLVTRVFKLLQNQIQQQTWTDEGTPSMRELRSALLEFACTHNLGNCSTTAMKLFDDWMASNGTQSLPTDVMTTVFKVGAKTDKGWSFLLGKYISIGSEAEKNKILEALASSEDVRKLYWLMKSSLNGDNFRTQKLSFIIRTVGRHFPGHLLAWDFVKENWNKLVQKFPLGSYTIQNIVAGSTYLFSTKTHLSEVQAFFENQSEATFRLRCVQEALEVIQLNIQWMEKNLKSLTWWL

SEQ ID NO: 10 - protein, 1011 aa, Homo sapiens
MIENSMFEEEPDVVDLAKEPCLHPLEPDEVEYEPRGSRLLVRGLGEHEMEEDEEDYESSAKLLGMSFMNRSSGLRNSATGYRQSPDGACSVPSARTMVVCAFVIVVAVSVIMVIYLLPRCTFTKEGCHKKNQSIGLIQPFATNGKLFPWAQIRLPTAVVPLRYELSLHPNLTSMTFRGSVTISVQALQVTWNIILHSTGHNISRVTFMSAVSSQEKQAEILEYAYHGQIAIVAPEALLAGHNYTLKIEYSANISSSYYGFYGFSYTDESNEKKYFAATQFEPLAARSAFPCFDEPAFKATFIIKIIRDEQYTALSNMPKKSSVVLDDGLVQDEFSESVKMSTYLVAFIVGEMKNLSQDVNGTLVSIYAVPEKIGQVHYALETTVKLLEFFQNYFEIQYPLKKLDLVAIPDFEAGAMENWGLLTFREETLLYDSNTSSMADRKLVTKIIAHELAHQWFGNLVTMKWWNDLWLNEGFATFMEYFSLEKIFKELSSYEDFLDARFKTMKKDSLNSSHPISSSVQSSEQIEEMFDSLSYFKGSSLLLMLKTYLSEDVFQHAVVLYLHNHSYASIQSDDLWDSFNEVTNQTLDVKRMMKTWTLQKGFPLVTVQKKGKELFIQQERFFLNMKPEIQPSDTSYLWHIPLSYVTEGRNYSKYQSVSLLDKKSGVINLTEEVLWVKVNINMNGYYIVHYADDDWEALIHQLKINPYVLSDKDRANLINNIFELAGLGKVPLKRAFDLINYLGNENHTAPITEALFQTDLIYNLLEKLGYMDLASRLVTRVFKLLQNQIQQQTWTDEGTPSMRELRSALLEFACTHNLGNCSTTAMKLFDDWMASNGTQSLPTDVMTTVFKVGAKTDKGWSFLLGKYISIGSEAEKNKILEALASSEDVRKLYWLMKSSLNGDNFRTQKLSFIIRTVGRHFPGHLLAWDFVKENWNKLVQKFPLGSYTVQNIVAGSTYLFSTKTHLSEVQAFFENQSEATFRLRCVQEALEVIQLNIQWMEKNLKSLTWWL

SEQ ID NO: 11 - protein, 1011 aa, Homo sapiens [sequence omitted]

SEQ ID NO: 12 - protein, 1006 aa, Homo sapiens
MFEEEPDVVDLAKEPCLHPLEPDEVEYEPRGSRLLVRGLGEHEMEEDEEDYESSAKLLGMSFMNRSSGLRNSATGYRQSPDGACSVPSARTMVVCAFVIVVAVSVIMVIYLLPRCTFTKEGCHKKNQSIGLIQPFATNGKLFPWAQIRLPTAVVPLRYELSLHPNLTSMTFRGSVTISVQALQVTWNIILHSTGHNISRVTFMSAVSSQEKQAEILEYAYHGQIAIVAPEALLAGHNYTLKIEYSANISSSYYGFYGFSYTDESNEKKYFAATQFEPLAARSAFPCFDEPAFKATFIIKIIRDEQYTALSNMPKKSSVVLDDGLVQDEFSESVKMSTYLVAFIVGEMKNLSQDVNGTLVSIYAVPEKIGQVHYALETTVKLLEFFQNYFEIQYPLKKLDLVAIPDFEAGAMENWGLLTFREETLLYDSNTSSMADRKLVTKIIAHELAHQWFGNLVTMKWWNDLWLNEGFATFMEYFSLEKIFKELSSYEDFLDARFKTMKKDSLNSSHPISSSVQSSEQIEEMFDSLSYFKGSSLLLMLKTYLSEDVFQHAVVLYLHNHSYASIQSDDLWDSFNEVTNQTLDVKRMMKTWTLQKGFPLVTVQKKGKELFIQQERFFLNMKPEIQPSDTSYLWHIPLSYVTEGRNYSKYQSVSLLDKKSGVINLTEEVLWVKVNINMNGYYIVHYADDDWEALIHQLKINPYVLSDKDRANLINNIFELAGLGKVPLKRAFDLINYLGNENHTAPITEALFQTDLIYNLLEKLGYMDLASRLVTRVFKLLQNQIQQQTWTDEGTPSMRELRSALLEFACTHNLGNCSTTAMKLFDDWMASNGTQSLPTDVMTTVFKVGAKTDKGWSFLLGKYISIGSEAEKNKILEALASSEDVRKLYWLMKSSLNGDNFRTQKLSFIIRTVGRHFPGHLLAWDFVKENWNKLVQKFPLGSYTIQNIVAGSTYLFSTKTHLSEVQAFFENQSEATFRLRCVQEALEVIQLNIQWMEKNLKSLTWWL

SEQ ID NO: 13 - protein, 1006 aa, Homo sapiens
MFEEEPDVVDLAKEPCLHPLEPDEVEYEPRGSRLLVRGLGEHEMEEDEEDYESSAKLLGMSFMNRSPGLRNSATGYRQSPDGACSVPSARTMVVCAFVIVVAVSVIMVIYLLPRCTFTKEGCHKKNQSIGLIQPFATNGKLFPWAQIRLPTAVVPLRYELSLHPNLTSMTFRGSVTISVQALQVTWNIILHSTGHNISRVTFMSAVSSQEKQAEILEYAYHGQIAIVAPEALLAGHNYTLKIEYSANISSSYYGFYGFSYTDESNEKKYFAATQFEPLAARSAFPCFDEPAFKATFIIKIIRDEQYTALSNMPKKSSVVLDDGLVQDEFSESVKMSTYLVAFIVGEMKNLSQDVNGTLVSIYAVPEKIGQVHYALETTVKLLEFFQNYFEIQYPLKKLDLVAIPDFEAGAMENWGLLTFREETLLYDSNTSSMADRKLVTKIIAHELAHQWFGNLVTMKWWNDLWLNEGFATFMEYFSLEKIFKELSSYEDFLDARFKTMKKDSLNSSHPISSSVQSSEQIEEMFDSLSYFKGSSLLLMLKTYLSEDVFQHAVVLYLHNHSYASIQSDDLWDSFNEVTNQTLDVKRMMKTWTLQKGFPLVTVQKKGKELFIQQERFFLNMKPEIQPSDTSYLWHIPLSYVTEGRNYSKYQSVSLLDKKSGVINLTEEVLWVKVNINMNGYYIVHYADDDWEALIHQLKINPYVLSDKDRANLINNIFELAGLGKVPLKRAFDLINYLGNENHTAPITEALFQTDLIYNLLEKLGYMDLASRLVTRVFKLLQNQIQQQTWTDEGTPSMRELRSALLEFACTHNLGNCSTTAMKLFDDWMASNGTQSLPTDVMTTVFKVGAKTDKGWSFLLGKYISIGSEAEKNKILEALASSEDVRKLYWLMKSSLNGDNFRTQKLSFIIRTVGRHFPGHLLAWDFVKENWNKLVQKFPLGSYTIQNIVAGSTYLFSTKTHLSEVQAFFENQSEATFRLRCVQEALEVIQLNIQWMEKNLKSLTWWL

SEQ ID NO: 14 - protein, 1006 aa, Homo sapiens
MFEEEPDVVDLAKEPCLHPLEPDEVEYEPRGSRLLVRGLGEHEMEEDEEDYESSAKLLGMSFMNRSSGLRNSATGYRQSPDGACSVPSARTMVVCAFVIVVAVSVIMVIYLLPRCTFTKEGCHKKNQSIGLIQPFATNGKLFPWAQIRLPTAVVPLRYELSLHPNLTSMTFRGSVTISVQALQVTWNIILHSTGHNISRVTFMSAVSSQEKQAEILEYAYHGQIAIVAPEALLAGHNYTLKIEYSANISSSYYGFYGFSYTDESNEKKYFAATQFEPLAARSAFPCFDEPAFKATFIIKIIRDEQYTALSNMPKKSSVVLDDGLVQDEFSESVKMSTYLVAFIVGEMKNLSQDVNGTLVSIYAVPEKIGQVHYALETTVKLLEFFQNYFEIQYPLKKLDLVAIPDFEAGAMENWGLLTFREETLLYDSNTSSMADRKLVTKIIAHELAHQWFGNLVTMKWWNDLWLNEGFATFMEYFSLEKIFKELSSYEDFLDARFKTMKKDSLNSSHPISSSVQSSEQIEEMFDSLSYFKGSSLLLMLKTYLSEDVFQHAVVLYLHNHSYASIQSDDLWDSFNEVTNQTLDVKRMMKTWTLQKGFPLVTVQKKGKELFIQQERFFLNMKPEIQPSDTSYLWHIPLSYVTEGRNYSKYQSVSLLDKKSGVINLTEEVLWVKVNINMNGYYIVHYADDDWEALIHQLKINPYVLSDKDRANLINNIFELAGLGKVPLKRAFDLINYLGNENHTAPITEALFQTDLIYNLLEKLGYMDLASRLVTRVFKLLQNQIQQQTWTDEGTPSMRELRSALLEFACTHNLGNCSTTAMKLFDDWMASNGTQSLPTDVMTTVFKVGAKTDKGWSFLLGKYISIGSEAEKNKILEALASSEDVRKLYWLMKSSLNGDNFRTQKLSFIIRTVGRHFPGHLLAWDFVKENWNKLVQKFPLGSYTVQNIVAGSTYLFSTKTHLSEVQAFFENQSEATFRLRCVQEALEVIQLNIQWMEKNLKSLTWWL

SEQ ID NO: 15 - protein, 1006 aa, Homo sapiens
MFEEEPDVVDLAKEPCLHPLEPDEVEYEPRGSRLLVRGLGEHEMEEDEEDYESSAKLLGMSFMNRSSGLRNSATGYRQSPDGACSVPSARTMVVCAFVIVVAVSVIMVIYLLPRCTFTKEGCHKKNQSIGLIQPFATNGKLFPWAQIRLPTAVVPLRYELSLHPNLTSMTFRGSVTISVQALQVTWNIILHSTGHNISRVTFMSAVSSQEKQAEILEYAYHGQIAIVAPEALLAGHNYTLKIEYSANISSSYYGFYGFSYTDESNEKKYFAATQFEPLAARSAFPCFDEPAFKATFIIKIIRDEQYTALSNMPKKSSVVLDDGLVQDEFSESVKMSTYLVAFIVGEMKNLSQDVNGTLVSIYAVPEKIGQVHYALETTVKLLEFFQNYFEIQYPLKKLDLVAIPDFEAGAMENWGLLTFREETLLYDSNTSSMADRKLVTKIIAHELAHQWFGNLVTMKWWNDLWLNEGFATFMEYFSLEKIFKELSSYEDFLDARFKTMKKDSLNSSHPISSSVQSSEQIEEMFDSLSYFKGSSLLLMLKTYLSEDVFQHAVVLYLHNHSYASIQSDDLWDSFNEVTNQTLDVKRMMKTWTLQKGFPLVTVQKKGKELFIQQERFFLNMKPEIQPSDTSYLWHIPLSYVTEGRNYSKYQSVSLLDKKSGVINLTEEVLWVKVNINMNGYYIVHYADDDWEALIHQLKINPYVLSDKDRANLINNIFELAGLGKVPLKRAFDLINYLGNENHTTPITEALFQTDLIYNLLEKLGYMDLASRLVTRVFKLLQNQIQQQTWTDEGTPSMRELRSALLEFACTHNLGNCSTTAMKLFDDWMASNGTQSLPTDVMTTVFKVGAKTDKGWSFLLGKYISIGSEAEKNKILEALASSEDVRKLYWLMKSSLNGDNFRTQKLSFIIRTVGRHFPGHLLAWDFVKENWNKLVQKFPLGSYTIQNIVAGSTYLFSTKTHLSEVQAFFENQSEATFRLRCVQEALEVIQLNIQWMEKNLKSLTWWL

SEQ ID NO: 16 - protein, 894 aa, Homo sapiens
PRCTFTKEGCHKKNQSIGLIQPFATNGKLFPWAQIRLPTAVVPLRYELSLHPNLTSMTFRGSVTISVQALQVTWNIILHSTGHNISRVTFMSAVSSQEKQAEILEYAYHGQIAIVAPEALLAGHNYTLKIEYSANISSSYYGFYGFSYTDESNEKKYFAATQFEPLAARSAFPCFDEPAFKATFIIKIIRDEQYTALSNMPKKSSVVLDDGLVQDEFSESVKMSTYLVAFIVGEMKNLSQDVNGTLVSIYAVPEKIGQVHYALETTVKLLEFFQNYFEIQYPLKKLDLVAIPDFEAGAMENWGLLTFREETLLYDSNTSSMADRKLVTKIIAHELAHQWFGNLVTMKWWNDLWLNEGFATFMEYFSLEKIFKELSSYEDFLDARFKTMKKDSLNSSHPISSSVQSSEQIEEMFDSLSYFKGSSLLLMLKTYLSEDVFQHAVVLYLHNHSYASIQSDDLWDSFNEVTNQTLDVKRMMKTWTLQKGFPLVTVQKKGKELFIQQERFFLNMKPEIQPSDTSYLWHIPLSYVTEGRNYSKYQSVSLLDKKSGVINLTEEVLWVKVNINMNGYYIVHYADDDWEALIHQLKINPYVLSDKDRANLINNIFELAGLGKVPLKRAFDLINYLGNENHTAPITEALFQTDLIYNLLEKLGYMDLASRLVTRVFKLLQNQIQQQTWTDEGTPSMRELRSALLEFACTHNLGNCSTTAMKLFDDWMASNGTQSLPTDVMTTVFKVGAKTDKGWSFLLGKYISIGSEAEKNKILEALASSEDVRKLYWLMKSSLNGDNFRTQKLSFIIRTVGRHFPGHLLAWDFVKENWNKLVQKFPLGSYTIQNIVAGSTYLFSTKTHLSEVQAFFENQSEATFRLRCVQEALEVIQLNIQWMEKNLKSLTWWL

SEQ ID NO: 17 - protein, 951 aa, Homo sapiens
KLLGMSFMNRSSGLRNSATGYRQSPDGACSVPSARTMVVCAFVIVVAVSVIMVIYLLPRCTFTKEGCHKKNQSIGLIQPFATNGKLFPWAQIRLPTAVVPLRYELSLHPNLTSMTFRGSVTISVQALQVTWNIILHSTGHNISRVTFMSAVSSQEKQAEILEYAYHGQIAIVAPEALLAGHNYTLKIEYSANISSSYYGFYGFSYTDESNEKKYFAATQFEPLAARSAFPCFDEPAFKATFIIKIIRDEQYTALSNMPKKSSVVLDDGLVQDEFSESVKMSTYLVAFIVGEMKNLSQDVNGTLVSIYAVPEKIGQVHYALETTVKLLEFFQNYFEIQYPLKKLDLVAIPDFEAGAMENWGLLTFREETLLYDSNTSSMADRKLVTKIIAHELAHQWFGNLVTMKWWNDLWLNEGFATFMEYFSLEKIFKELSSYEDFLDARFKTMKKDSLNSSHPISSSVQSSEQIEEMFDSLSYFKGSSLLLMLKTYLSEDVFQHAVVLYLHNHSYASIQSDDLWDSFNEVTNQTLDVKRMMKTWTLQKGFPLVTVQKKGKELFIQQERFFLNMKPEIQPSDTSYLWHIPLSYVTEGRNYSKYQSVSLLDKKSGVINLTEEVLWVKVNINMNGYYIVHYADDDWEALIHQLKINPYVLSDKDRANLINNIFELAGLGKVPLKRAFDLINYLGNENHTAPITEALFQTDLIYNLLEKLGYMDLASRLVTRVFKLLQNQIQQQTWTDEGTPSMRELRSALLEFACTHNLGNCSTTAMKLFDDWMASNGTQSLPTDVMTTVFKVGAKTDKGWSFLLGKYISIGSEAEKNKILEALASSEDVRKLYWLMKSSLNGDNFRTQKLSFIIRTVGRHFPGHLLAWDFVKENWNKLVQKFPLGSYTIQNIVAGSTYLFSTKTHLSEVQAFFENQSEATFRLRCVQEALEVIQLNIQWMEKNLKSLTWWL

SEQ ID NO: 18 - protein, 894 aa, Homo sapiens; identical to SEQ ID NO: 16

SEQ ID NO: 19 - protein, 894 aa, Homo sapiens
PRCTFTKEGCHKKNQSIGLIQPFATNGKLFPWAQIRLPTAVVPLRYELSLHPNLTSMTFRGSVTISVQALQVTWNIILHSTGHNISRVTFMSAVSSQEKQAEILEYAYHGQIAIVAPEALLAGHNYTLKIEYSANISSSYYGFYGFSYTDESNEKKYFAATQFEPLAARSAFPCFDEPAFKATFIIKIIRDEQYTALSNMPKKSSVVLDDGLVQDEFSESVKMSTYLVAFIVGEMKNLSQDVNGTLVSIYAVPEKIGQVHYALETTVKLLEFFQNYFEIQYPLKKLDLVAIPDFEAGAMENWGLLTFREETLLYDSNTSSMADRKLVTKIIAHELAHQWFGNLVTMKWWNDLWLNEGFATFMEYFSLEKIFKELSSYEDFLDARFKTMKKDSLNSSHPISSSVQSSEQIEEMFDSLSYFKGSSLLLMLKTYLSEDVFQHAVVLYLHNHSYASIQSDDLWDSFNEVTNQTLDVKRMMKTWTLQKGFPLVTVQKKGKELFIQQERFFLNMKPEIQPSDTSYLWHIPLSYVTEGRNYSKYQSVSLLDKKSGVINLTEEVLWVKVNINMNGYYIVHYADDDWEALIHQLKINPYVLSDKDRANLINNIFELAGLGKVPLKRAFDLINYLGNENHTTPITEALFQTDLIYNLLEKLGYMDLASRLVTRVFKLLQNQIQQQTWTDEGTPSMRELRSALLEFACTHNLGNCSTTAMKLFDDWMASNGTQSLPTDVMTTVFKVGAKTDKGWSFLLGKYISIGSEAEKNKILEALASSEDVRKLYWLMKSSLNGDNFRTQKLSFIIRTVGRHFPGHLLAWDFVKENWNKLVQKFPLGSYTIQNIVAGSTYLFSTKTHLSEVQAFFENQSEATFRLRCVQEALEVIQLNIQWMEKNLKSLTWWL

SEQ ID NO: 20 - protein, 894 aa, Homo sapiens
PRCTFTKEGCHKKNQSIGLIQPFATNGKLFPWAQIRLPTAVVPLRYELSLHPNLTSMTFRGSVTISVQALQVTWNIILHSTGHNISRVTFMSAVSSQEKQAEILEYAYHGQIAIVAPEALLAGHNYTLKIEYSANISSSYYGFYGFSYTDESNEKKYFAATQFEPLAARSAFPCFDEPAFKATFIIKIIRDEQYTALSNMPKKSSVVLDDGLVQDEFSESVKMSTYLVAFIVGEMKNLSQDVNGTLVSIYAVPEKIGQVHYALETTVKLLEFFQNYFEIQYPLKKLDLVAIPDFEAGAMENWGLLTFREETLLYDSNTSSMADRKLVTKIIAHELAHQWFGNLVTMKWWNDLWLNEGFATFMEYFSLEKIFKELSSYEDFLDARFKTMKKDSLNSSHPISSSVQSSEQIEEMFDSLSYFKGSSLLLMLKTYLSEDVFQHAVVLYLHNHSYASIQSDDLWDSFNEVTNQTLDVKRMMKTWTLQKGFPLVTVQKKGKELFIQQERFFLNMKPEIQPSDTSYLWHIPLSYVTEGRNYSKYQSVSLLDKKSGVINLTEEVLWVKVNINMNGYYIVHYADDDWEALIHQLKINPYVLSDKDRANLINNIFELAGLGKVPLKRAFDLINYLGNENHTAPITEALFQTDLIYNLLEKLGYMDLASRLVTRVFKLLQNQIQQQTWTDEGTPSMRELRSALLEFACTHNLGNCSTTAMKLFDDWMASNGTQSLPTDVMTTVFKVGAKTDKGWSFLLGKYISIGSEAEKNKILEALASSEDVRKLYWLMKSSLNGDNFRTQKLSFIIRTVGRHFPGHLLAWDFVKENWNKLVQKFPLGSYTVQNIVAGSTYLFSTKTHLSEVQAFFENQSEATFRLRCVQEALEVIQLNIQWMEKNLKSLTWWL

SEQ ID NO: 21 - protein, 6 aa, Homo sapiens
VYIHPF

SEQ ID NO: 22 - protein, 6 aa, Artificial sequence
  Peptide derived from angiotensin IV
XXXHXX

SEQ ID NO: 23 - protein, 6 aa, Artificial sequence
  Peptide derived from angiotensin IV
XYIHPF

SEQ ID NO: 24 - protein, 6 aa, Artificial sequence
  Peptide derived from angiotensin IV
XYIHPF

SEQ ID NO: 25 - protein, 6 aa, Artificial sequence
  Peptide derived from angiotensin IV
XYIHPX

SEQ ID NO: 26 - protein, 6 aa, Artificial sequence
  Peptide derived from angiotensin IV
XYIHPX

SEQ ID NO: 27 - protein, 6 aa, Artificial Sequence
  Peptide derived from angiotensin IV
VYIHXF

SEQ ID NO: 28 - protein, 5 aa, Artificial Sequence
  Peptide derived from angiotensin IV
XYIXF

SEQ ID NO: 29 - protein, 5 aa, Artificial Sequence
  Peptide derived from angiotensin IV
XYIXF

(19) United States
(12) Patent Application Publication
(10) Pub. No.: US 2012/0115163 A1
(43) Pub. Date: May 10, 2012

(54) MARKED PEPTIDES AND USE THEREOF FOR ASSAYING CIRCULATING IRAP

(75) Inventors: Serge Bottari, Biviers (FR); Georges Vauquelin, Limal (BE); Dirk Tourwe, Itterbeek (BE)

(73) Assignees: VRIJE UNIVERSITEIT BRUSSEL, Elsene (BE); UNIVERSITE JOSEPH FOURIER, Grenoble Cedex 09 (FR)

(21) Appl. No.: 13/321,576

(22) PCT Filed: May 19, 2010
(86) PCT No.: PCT/FR2010/050967

§ 371 Date: Jan. 26, 2012

(30) Foreign Application Priority Data
May 20, 2009 (FR) 09/53392

(51) Int. Cl. G01N 33/573; C07K 2/00; C07K 7/06; C12Q 1/37

(52) U.S. Cl. 435/7.1; 435/24; 530/300; 530/329

(57) ABSTRACT

A method for the assay of the circulating extracellular portion of the IRAP protein (“insulin responsive aminopeptidase”) includes at least one stage of quantitative assay of the purified, secreted, extracellular portion of IRAP, via at least one labelled peptide, the labelled peptide interacting specifically with the extracellular portion of IRAP.

The present invention relates to labelled peptides and their use for the assay of circulating IRAP protein.

The TRAP protein (Insulin-Regulated AminoPeptidase; EC 3.4.11.3) also known as Placental Leucine AminoPeptidase (P-LAP) and Leucine-cystinyl aminopeptidase (L-CAP) is a transmembrane zinc metalloproteinase protein which exists in three isoforms (Swiss-Prot: Q9UIQ6:1, 2 and 3; represented respectively by SEQ ID NO: 1 to 3).

When it is inserted into the plasma membrane, in its native form, its extracellular domain can be cleaved and secreted.

The secreted part of an unspecified isoform of the TRAP protein was assayed in the blood by an enzymatic method using a non-specific synthetic substrate, L-leucine-nitroanilide in the presence of methionine (Mizutani, S., Yoshino, M., and Oya, M. (1976) Clin Biochem 9(1), 16-18).

Using this method Mizutani, Oya and Tomoda detected a cystinyl-leucine aminopeptidase. This aminopeptidase is essentially of placental origin, and therefore called P-LAP (Tsujimoto, M., Mizutani, S., Adachi, H., Kimura, M., Nakazato, H., and Tomoda, Y. (1992) Arch Biochem Biophys 292(2), 388-392) and corresponds to the secreted domain of the protein. This enzyme degrades oxytocin (Naruki, M., et al. (1996) Peptides 17(2), 257-261), vasopressin (Wallis, M. G. et al. (2007) Am J Physiol Endocrinol Metab 293(4), E1092-1102), angiotensin II and III (Matsumoto, H., et al. (2000) Eur J Biochem 267(1), 46-52) as well as a series of other peptides (Albiston, A. L., et al. (2007) Pharmacol Ther 116(3), 417-427). The serous concentrations of this aminopeptidase have never been reported in humans. At the time of its cloning, it appeared that P-LAP corresponds to the TRAP protein as well as to angiotensin IV receptor (Keller, S. R., et al. (1995) J Biol Chem 270(40), 23612-23618; Rogi, T., et al. (1996) J Biol Chem 271(1), 56-61; Albiston, A. L., and al (2001) J Biol Chem 276(52), 48623-48626).

International Application WO 2005/038462 describes a reagent for diagnosis and/or prognostic evaluation of carcinoma, comprising an anti-P-LAP polyclonal antibody, obtained by immunization with the whole P-LAP protein. Given the homology between the different aminopeptidases, the antibody is probably not specific to IRAP and should also recognize other aminopeptidases. It should not therefore make it possible to diagnose a pathology linked in a precise manner to a modification of the expression or plasmatic concentration of IRAP. A relationship has been demonstrated between the IRAP protein and the development of chemoresistance to anticancer drugs (Kondo C. et al., Int J. Cancer, 118, 1390-1394, 2006). According to this article, TRAP in fact reduces sensitivity to anticancer drugs by inhibiting the expression of the apoptosis-induction factor and increases expression of the apoptosis-inhibition factor.

The works of Lukaszuk et al. (J. Med. Chem. 2008, 51, pp: 2291-2296) have shown that peptides derived from angiotensin IV were capable of binding specifically to the TRAP protein located at the surface of the cells.

At present, no method exists making it possible to measure and quantify the IRAP protein in its native form, i.e. not cleaved and anchored in the plasma membrane.

The extracellular domain of IRAP is cleaved by metalloproteases probably belonging to the ADAM family including ADAM9 and ADAM12 (Ito, N., et al. (2004) Biochem Biophys Res Commun 314(4), 1008-1013) and released into the blood circulation. ADAM9 (MDC9) is expressed in particular in the brain, the liver, the heart, the kidneys and the lungs (Hotoda, N., et al. (2002) Biochem Biophys Res Commun 293(2), 800-805) and several other members of this family are also expressed in the muscle and adipose tissue.

At present, the only relationships established between the concentration of the extracellular domain of TRAP in a biological medium and a pathology concern severe preeclampsia as well as the risk of premature labour.

Therefore it appears necessary to assay the circulating extracellular domain of the IRAP protein accurately and quantitatively.

At present, the circulating TRAP protein has never been assayed quantitatively in serum.

The problem to be solved is even more complicated than that of assaying IRAP in its native form given, on the one hand, the presence, in the serum or the plasma, of numerous elements in particular proteases capable of degrading the potential IRAP ligands and, on the other hand, the need to utilize relatively demanding means.

There is therefore a need for a means of assaying the circulating TRAP protein in serum or plasma by means of compounds capable of resisting degradations caused by the proteases contained in the serum or the plasma.

There is therefore also a need for specific, stable compounds which can be used for the implementation of a quantitative and reliable assay of the IRAP protein, in a biological sample containing no cells.

Thus, the purpose of the invention is to provide a reliable, specific and quantitative method making it possible to measure the quantity of circulating IRAP protein. Another subject of the invention is to provide ligands of circulating TRAP allowing its detection.

Thus, the invention relates to a method for the assay of the circulating extracellular portion of the IRAP protein (“insulin responsive aminopeptidase”) comprising at least one stage of quantitative assay of said purified, secreted extracellular portion of TRAP, by means of at least one labelled peptide, said labelled peptide interacting specifically with said extracellular portion of TRAP,
said labelled peptide being preferentially labelled with at least one radioactive isotope,
said labelled peptide being a ligand of TRAP, and in particular
   either a labelled substrate of the IRAP protein,
   or a labelled peptide inhibiting the TRAP protein,
said peptide being optionally modified by the presence of at least one non-natural amino acid, and/or at least one β-homo amino acid, natural or non-natural,
provided that said unmodified labelled peptide is different from angiotensin IV, and in particular that said unmodified labelled peptide is different from the sequence SEQ ID NO 21 (Val-Tyr-Ile-His-Pro-Phe), and
   provided that said modified peptide, labelled with an iodine atom ¹²⁵I, is different from the sequence Nle-Tyr-Ile-His-Pro-Phe (SEQ ID NO 23).

The invention relates to a method for the assay of the circulating extracellular portion of the IRAP protein (“insulin responsive aminopeptidase”) comprising at least one stage of quantitative assay of said purified, secreted extracellular portion of TRAP, by means of at least one modified and labelled peptide, said labelled peptide interacting specifically with said extracellular portion of TRAP,
said peptide being modified by the presence of at least one non-natural amino acid, and/or at least one β-homo amino acid, natural or non-natural,
said labelled peptide being preferentially labelled with at least one radioactive isotope, provided that
   said peptide is not an antibody and
   said modified peptide, labelled with an iodine atom ¹²⁵I, is different from the sequence Nle-Tyr-Ile-His-Pro-Phe (SEQ ID NO 23).

The present invention is based on the inventors' unexpected finding that labelled peptides allow an effective quantitative detection of the extracellular portion of circulating TRAP.

The “extracellular portion” of the IRAP protein is defined in the invention as the domain, or the sequence of amino acids, of the IRAP protein which is exposed to the outside of the cell whilst the IRAP protein is anchored in the plasma membrane of the cells.

The “circulating extracellular portion” of the IRAP protein is defined in the invention as the extracellular portion of the IRAP protein as defined above which is no longer covalently bound to the transmembrane part of the IRAP protein. This extracellular portion is described as circulating because, after being cleaved, it is found in the extracellular medium, and can in particular be found in the plasma, serum, lymph, cerebrospinal fluid or also urine.

The circulating extracellular portion of IRAP is also called the secreted extracellular portion of IRAP when it is found in the general circulation of the organism, i.e. the plasma, serum, lymph, cerebrospinal fluid or urine.

The terms “circulating extracellular portion of IRAP” and “extracellular portion of circulating IRAP” hereafter and above, both refer to the extracellular domain of the IRAP protein which is no longer associated with the membrane part of IRAP, and therefore is no longer associated with the cell membrane.

The IRAP protein to which the invention relates is represented by the amino acid sequence SEQ ID NO 1.

By “peptide” is meant any peptide chain corresponding to a covalent sequence of at least two amino acids, or amino acid derivatives, bound by an amide function (—CO—NH—), said amino acids being natural or non-natural and said amino acid derivatives being natural or non-natural amino acid derivatives.

By “labelled peptide”, is meant in the invention a peptide as defined above possessing a molecule or an atom allowing its detection other than by standard methods for detecting proteins or peptides, i.e. methods other than immunological methods. Thus, the labelled peptides according to the invention can be coupled either to fluorescent molecules (cyanines, coumarins, rhodamines, xanthenes, quantum dots (nanocrystals of semiconductor materials) etc.) can either contain at least one, i.e. one or more, radioactive atom(s), said radioactive atom also being called a radioactive isotope. Among the radioactive isotopes capable of being used in the invention, there can be mentioned, tritium (³H), carbon 14 (¹⁴C), iodine 131 (¹³¹I), iodine 125 (¹²⁵I) or also phosphate 32 or 33 (³²P or ³³P).

Hereafter the peptides according to the invention are labelled using one or more iodine 125 atoms (¹²⁵I), preferentially by a single iodine 125 atom (¹²⁵I).

The radioactive isotopes are capable of disintegrating and emitting particles in the form of radiation, said radiation being measurable, for example by means of scintillation counters, or not, and said radiation being proportional to the quantity of isotope.

In the assay described in the invention, the quantification of the extracellular portion of IRAP is carried out starting from the extracellular portion of IRAP which has been purified.

The methods for purification of the circulating TRAP protein can be any method for purification of proteins known to a person skilled in the art. A preferred, but non-limitative, method is an immunological method and in particular the immunoprecipitation or the immunocapture of the circulating IRAP protein, by means of a specific antibody directed against the extracellular portion of the TRAP protein. The immunological method of immunocapture of the extracellular portion of circulating TRAP is described in the examples. In the invention by “quantitative assay” is meant the action which consists of accurately determining the quantity of circulating IRAP protein contained in a biological sample from an individual or animal.

The term “said labelled peptide interacting specifically with said extracellular portion of IRAP”, used in the invention, signifies that at least one labelled peptide according to the invention and the purified extracellular portion of circulating IRAP form a high-affinity complex. The order of magnitude of the affinity between said peptides and said circulating extracellular portion of IRAP is comprised between approximately 10⁻¹⁰ M and 10⁻⁵ M, preferentially between approximately 0.1 nM and approximately 100 nM. The term approximately is used to cover the variability in the measurement of said affinity, said variation being generally from 5 to 10% measurement error.

The intensity of the interaction is measured for example as described in Lukaszuk et al. (J. Med. Chem. 2008, 51, pp: 2291-2296).

The interaction is described as specific, which signifies that the labelled peptide according to the invention is capable of forming a complex with the circulating extracellular portion of the IRAP protein, but said labelled peptide is not capable of forming a complex of the same affinity, at the same concentrations, with another protein possessing an amino acid sequence different from the amino acid sequence of said extracellular portion of TRAP.

The labelled peptide of the invention is a ligand of IRAP, and in particular either a labelled substrate of the IRAP protein, or a labelled IRAP protein-inhibiting peptide, or any peptide the affinity of which for the extracellular portion of circulating IRAP is approximately less than or equal to 10 μM (≦10 nM).

According to the invention, the peptide defined above can be modified by the presence of at least one non-natural amino acid, and/or at least one natural or non-natural β-homo amino acid.

By “non-natural amino acid”, is meant in the invention amino acids which are not found naturally in proteins when they are synthesized in a living organism, or an acellular system using the natural protein synthesis machinery (mRNA, tRNA, ribosomes etc.). The natural amino acids are the 20 amino acids known to a person skilled in the art, the correspondence of which with the nucleotide triplets is given by the genetic code.

The non-natural amino acids used in the invention are in particular, but are not limited to, amino acids derived from leucine such as cycloleucine (Cle), norleucine (Nle) or tert-leucine (Tle).

The term “β-homo amino acid”, defines in the invention an amino acid possessing an additional carbon. In other words, the amino acids correspond to 2-(or α)amino 2-[Side chain (R)]acetic acids, and the β homo amino acids correspond to 3-(or β homo) 2-[Side chain (R)] amino propanoic acids (β²homo amino acid), or 3-(or β homo) 3-[Side chain (R)] amino propanoic acids (β³homo amino acid).

The formulae of the β²homo amino acids and the β³homo amino acids are as follows:

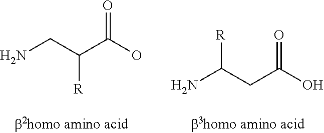

where R represents any one of the residues determining the amino acids.

The invention does not relate to the use of an unmodified labelled peptide which would be angiotensin IV, and in particular human Angiotensin IV of sequence SEQ ID NO: 21 (Val-Tyr-Ile-His-Pro-Phe).

The invention does not relate to the use of a modified peptide, labelled with an iodine ¹²⁵I atom, corresponding to Angiotensin IV where the valine in position 1 is replaced by an Nle and represented by the sequence SEQ ID NO: 23 (Nle-Tyr-Ile-His-Pro-Phe).

Above and hereafter, by convention, the specific sequences will make apparent the natural amino acids, the non-natural amino acids, and the β² or β³ derivatives of said natural or non-natural amino acids. On the other hand, the labelling does not appear in the sequence, and therefore the labelled peptide of sequence X is represented by the unlabelled sequence SEQ ID NO X, and the type of labelling is specified. For example the peptide X labelled with radioactive iodine is indicated as follows: peptide of sequence SEQ ID NO X labelled with iodine ¹²⁵I.

Advantageously, a subject of the invention is a method for the assay of the circulating extracellular portion of IRAP as defined above, where said labelled peptide is not modified, provided that said unmodified labelled peptide is different from angiotensin IV, and in particular that said unmodified labelled peptide is different from the sequence SEQ ID NO 21 (Val-Tyr-Ile-His-Pro-Phe).

These unmodified labelled peptides have the advantage of being similar or equivalent to the unlabelled peptides, and therefore of having a strong affinity for the extracellular portion of the circulating IRAP protein.

According to another advantageous embodiment, the invention relates to a method for the assay of the circulating extracellular portion of TRAP as defined above, where said labelled peptide is modified by the presence of at least one non-natural amino acid, and/or at least one β-homo amino acid, natural or non-natural,

said modified labelled peptide interacting specifically with said extracellular portion of IRAP, provided that said modified peptide, labelled with an iodine ¹²⁵I atom, is different from the sequence Nle-Tyr-Ile-His-Pro-Phe (SEQ ID NO 23).

These modified labelled peptides have the advantage of having a strong affinity for the extracellular portion of the circulating IRAP protein, and of having a high degree of stability, in particular in a sample containing numerous proteases or peptidases.

The stability of the peptides according to the invention can in particular be measured by evaluating the inhibiting properties exerted by said peptides on the aminopeptidase activity of IRAP. This stability measurement is illustrated in the examples.

In an advantageous embodiment, the invention also relates to a method for the assay of the circulating extracellular portion of IRAP as defined previously, comprising at least:
one stage of purification of said circulating extracellular portion of IRAP, and
one stage of quantification of said circulating extracellular portion purified in the previous stage, by means of at least one labelled modified peptide.

Thus, the method according to the invention comprises at least one stage of purification of the extracellular portion of IRAP in a biological sample. This stage of purification is followed by a stage of quantification of said extracellular portion of TRAP purified in the previous stage. This second stage is implemented by means of at least one modified peptide as defined previously.

As indicated previously, the purification of the circulating extracellular portion of IRAP can be implemented by an immunological method, i.e. a method using an antibody directed against the IRAP protein, in particular immunoprecipitation or immunocapture. This method is specific when it uses a specific antibody which recognizes only the extracellular portion of the IRAP protein, and makes it possible to obtain an extracellular portion of the pure IRAP protein. The degree of contamination by other proteins can be evaluated by a standard protein separation method (SDS-PAGE) coupled with a protein detection method (silver staining, colloidal Coomassie blue staining etc.), methods known to a person skilled in the art. The immunoprecipitation or the immunocapture allows purification to a very widely acceptable degree (>90%), and in particular makes it possible to isolate a protein from its environment, for example from the contaminating proteases.

In another advantageous embodiment, the invention relates to a method for the assay of the circulating extracellular portion of IRAP as defined previously, where
said labelled IRAP protein-inhibiting peptide is modified and is chosen from: labelled and modified Angiotensin IV and labelled and modified LVV-hemorphin-7, or
said labelled substrate of the IRAP protein is modified and is chosen from: labelled and modified [Arg]-vasopressin, labelled and modified Oxytocin, labelled and modified Met-/Leu-enkephalin, labelled and modified Somatostatin, labelled and modified CCK-8, labelled and modified Neurokinin A, labelled and modified Neuromedin B, labelled and modified Lys-bradykinin and labelled and modified Dynorphin A.

The labelled modified peptides capable of being used in the invention correspond either to substrates of the TRAP protein, or to IRAP protein-inhibiting peptides.

The inhibiting peptides are chosen from LVV-hemorphin-7 or angiotensin IV. These inhibiting peptides are modified and labelled.

The IRAP protein is a protease, it is therefore capable of cleaving proteins, said proteins being substrates of IRAP. The proteins mentioned above as TRAP substrates are therefore proteins capable of being cleaved by IRAP.

Like any enzyme, the catalytic activity of TRAP can be blocked by compounds called inhibitors. Generally inhibitors bind to the active site of an enzyme, are not metabolized by said enzyme, and prevent it from accessing its substrates. This kind of inhibition is a competitive inhibition. LVV-hemorphin-7 or angiotensin IV are peptides inhibiting the activity of IRAP.

In another advantageous embodiment, the invention relates to a method for the assay of the circulating extracellular portion of IRAP as defined above, where said purification of the circulating part of the IRAP protein is carried out by means of at least one antibody specific to said extracellular portion of IRAP, in particular by immunoprecipitation or immunocapture. Thus, in order to purify the extracellular portion of circulating IRAP in a biological sample, the invention proposes to use an antibody specifically recognizing the extracellular portion of IRAP, and not recognizing the transmembrane or intracellular portion of said IRAP protein. This method of purification can be an immunoprecipitation where a specific antibody of the extracellular portion of IRAP is attached to polymer beads of sugar (agarose, sepharose). The method of purification can also be an immunocapture within the context of an ELISA or a Radioimmunometric assay (RIMA) where said specific antibodies are fixed to a support, in particular the wall of a tube, and immobilize IRAP on the latter.

Advantageously, at least one of the following products can be added to the sample of serum, plasma, lymph, cerebrospinal fluid or urine in which the concentration of the extracellular portion of TRAP must be measured:
cation chelators such as: EDTA, EGTA, 1,10-orthophenantroline, dimercaprol, lipoic acid, BAPTA (1,2-bis(o-aminophenoxy)ethane-N,N,N′,N′-tetraacetic acid), DTPA (diethylene triamine pentaacetic acid), DMPS (2,3-dimercapto-1-propanesulphonic acid), DMSA (dimercaptosuccinic acid), penicillamine, deferroxamine, sulphosalicylic acid, citric acid, oxalic acid, tartaric acid, N,N-dimethyldecylamine N-oxide, nitrilotriacetic acid, pyromellitic acid, EDTMP (ethylenediamine tetra(methylene phosphonic acid)), ferric ferrocyanide, 2,2′-bipyridine, N,N′,N′-tris(2-pyridylmethyl)-cis,cis-1,3,5,-triaminocyclohexane (tachpyr) and its analogues, azobenzene and its analogues, diethyldithiocarbamate, TPEN (N,N,N′,N′-tetrakis(2-pyridylmethyl)ethylenediamine), 1,2-cyclohexylenedinitrilotetraacetic acid, maltol, thiomaltol, ethylmaltol, ethylthiomaltol, 2-mercaptopyridine oxide and other bidentate chelators,
protease and peptidase inhibitors such: AEBSF (4-(2-Aminoethyl)benzenesulphonyl fluoride hydrochloride), 6-aminohexanoic acid, antipain, aprotinin, benzamidine, bestatin, chymostatin, E-64, leupeptin, pepstatin, phosphoramidon, trypsin inhibitor, Diisopropyl fluorophosphate, PMSF, p-chloromercuribenzoic acid, diethyl pyrocarbonate, 2-Mercaptoethylamine, Apstatin, Phebestin, Bromoenol lactone, Ectoine (1,4,5,6-tetrahydro-2-methyl-4-pirymidine carboxylic acid), N-Acetyl-eglin C, Gabexate mesylate, N-Tosyl-L-phenylalanine chloromethyl ketone, Na-T-Boc-Deacetylleupeptin, 3,4-Dichloroisocoumarin, Amastatin, (2S,3R)-3-Amino-2-hydroxy-4-(4-nitro-phenyl)butanoyl-L-leucine, Actinonin, Epiamastatin, N-[(2S)-(Methoxycarbonylmethyl)-4-methylpentanoyl]-L-tryptophan-methylamide, oxidised 2,2′-Dipyridyl, L-Leucinethil, Epibestatin
other compounds: methionine, dithiotreitol, dithioerythritol, B-mercaptoethanol, TCEP (tris(2-carboxyethyl)phosphine), cysteine, N-ethylmaleimide, mercaptoethylamine, non-ionic detergents (Triton X-100, Tween 20 etc.)

All these products are known to a person skilled in the art and can be used at standard concentrations.

In another advantageous embodiment, the invention relates to a method for the assay of the circulating extracellular portion of TRAP defined previously, where said IRAP protein is represented by
the IRAP protein of sequence SEQ ID NO 1, or
a protein homologous to the IRAP protein exhibiting at least 80% sequence identity with the sequence SEQ ID NO 1, provided that said homologous protein is capable of interacting specifically with the IRAP ligands or the IRAP inhibiting peptides as defined previously, or
an isoform of the IRAP protein, said isoform being the product of the alternative splicing of the RNA encoding the IRAP protein SEQ ID NO 1, provided that said isoform is capable of interacting specifically with the IRAP ligands or the TRAP inhibiting peptides as defined previously, said isoform of the IRAP protein SEQ ID NO 1 being in particular represented by the proteins SEQ ID NO 2 or 3.

In the invention, the homologues of the IRAP protein correspond to proteins possessing a sequence homologous to the sequence SEQ ID NO 1 but possessing amino acid substitutions. The isoforms of the IRAP protein defined in the invention correspond to products of the alternative splicing of the product of the gene encoding the IRAP protein, so that the protein which results possesses a shorter sequence than the IRAP protein SEQ ID NO 1. The preferred isoforms of the invention correspond to truncated proteins in the amino-terminal part of several amino acids.

Another advantageous embodiment of the invention relates to a method of assay defined previously, where
said variants of the IRAP protein possess an amino acid sequence chosen from the sequences SEQ ID NO 4 to SEQ ID NO 7, and
said isoforms of the IRAP protein possess an amino acid sequence chosen from the sequences SEQ ID NO 8 to SEQ ID NO 15.

Also, according to another advantageous embodiment, the invention relates to a method of assay as defined previously, in which said circulating extracellular portion of the IRAP protein possesses an amino acid sequence represented by the sequences chosen from the following sequences: SEQ ID NO 16, SEQ ID NO 17, SEQ ID NO 18, SEQ ID NO 19 and SEQ ID NO 20.

Another advantageous embodiment of the invention relates to a method of assay defined previously, where said labelled modified peptide is labelled with at least one tritium atom or at least one atom of iodine ¹²⁵I, i.e. one iodine atom or more, preferentially a single atom of iodine ¹²⁵I.

In an advantageous embodiment, the invention also relates to a method of assay defined previously, where said labelled modified peptide is represented by the following general formula (III):

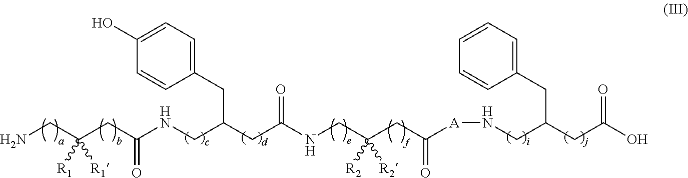

where a, b, c, d, e, f, g, h, i and j can be equal to 0 or 1, so that (a+b)≦1, (c+d)≦1, (e+f)≦1, (g+h)≦1 and (i,j)≦1, (a,b), (c,d), (e,f), (g,h) and (i,j) being independent of each other,
where (R₁, R₁′) are such that:
R₁ is chosen from: a —CH(CH₃)₂ group, a —CH₂—CH(CH₃)₂ group, a —CH(CH₃)—CH₂—CH₃ group, a —(CH₂)₃—CH₃ group and a C(CH₃)₃ group, and R₁′ is a hydrogen atom, or
R₁ and R₁′ form together with the carbon which carries them a cyclopentyl,
   where (R₂, R₂′) are such that:
if R₂ is chosen from: a —CH₂—CH(CH₃)₂ group, a —CH(CH₃)—CH₂—CH₃ group, a —(CH₂)₃—CH₃ group and a C(CH₃)₃ group, R₂′ is a hydrogen atom, or
R₂ and R₂′ form together with the carbon which carries them a cyclopentyl, the pairs (R₁, R₁′) and (R₂, R₂′) being chosen independently of each other,
   where A is chosen from the following groups

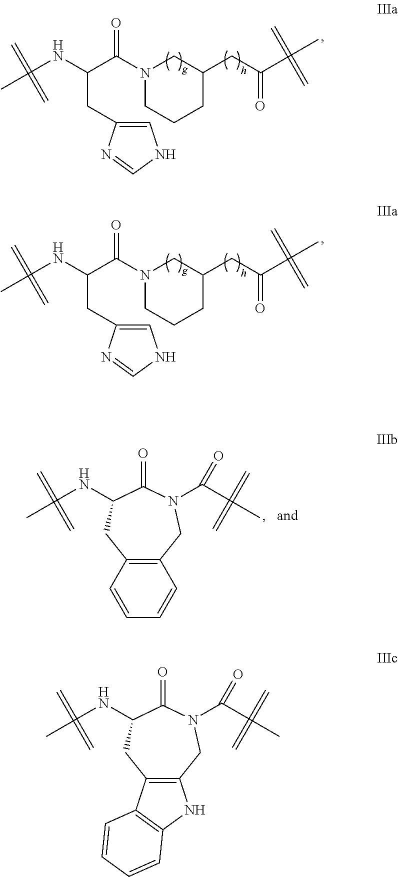

said labelled modified peptide being in the form of a racemate, any one of its enantiomers, or any one of the different tautomers corresponding to said racemates and enantiomers,
   provided that if A is represented by formula Ma and if a, b, c, d, e, f, g, h, i and j are equal to 0,
if (R₁, R₁′)═(—CH(CH₃)₂;H) then (R₂, R₂′) is different from (—CH(CH₃)—CH₂—CH₃;H), and

if (R₂, R₂′)═(—CH(CH₃)—CH₂—CH₃;H) then (R₁, R₁′) is different from (—CH(CH₃)₂;H) and provided that if said modified peptide is labelled with an iodine ¹²⁵I atom, if a, b, c, d, e, f, g, h, i and j are equal to 0 then (R₁, R₁′) is different from (—(CH₂)₃—CH₃;H).

In other words, the labelled modified peptide corresponds to one of the following three general formulae:

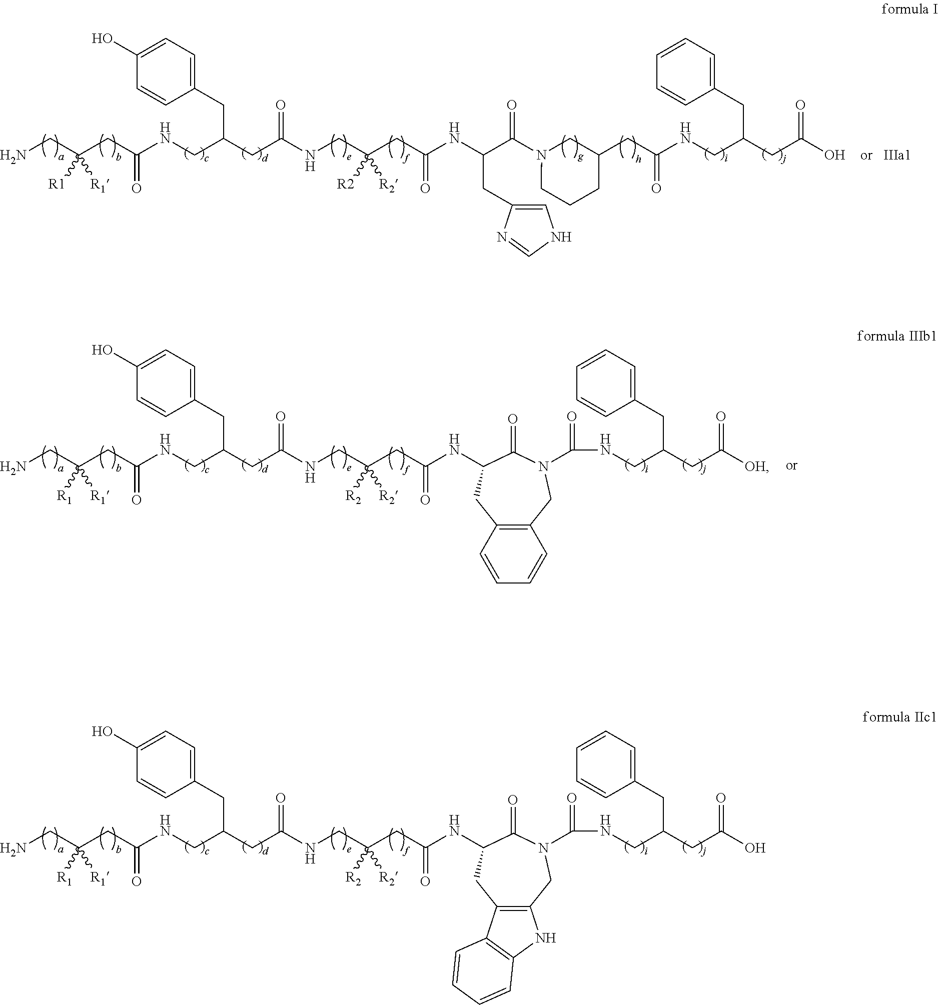

In an advantageous embodiment the invention also relates to a method of assay defined previously, where said labelled modified peptide is represented by general formula (I) below:

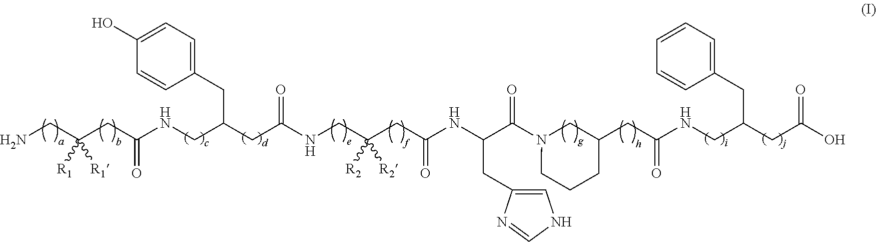

where a, b, c, d, e, f, g, h, i and j can be equal to 0 or 1, so that (a+b)≦1, (c+d)≦1, (e+f)≦1, (g+h)≦1 and (i,j)≦1, (a,b), (c,d), (e,f), (g,h) and (i,j) being independent of each other, where (R₁, R₁′) are such that:
R1 is chosen from: a —CH(CH₃)₂ group, a —CH₂—CH(CH₃)₂ group, a —CH(CH₃)—CH₂—CH₃ group, a —(CH₂)₃—CH₃ group and a C(CH₃)₃ group, and R1′ is a hydrogen atom, or

R1 and R1′ form together with the carbon which carries them a cyclopentyl,

where (R₂, R₂′) are such that:
if R2 is chosen from: a —CH₂—CH(CH₃)₂ group, a —CH(CH₃)—CH₂—CH₃ group, a —(CH₂)₃—CH₃ group and a C(CH₃)₃ group, R2′ is a hydrogen atom, or
R2 and R2′ form together with the carbon which carries them a cyclopentyl, the pairs (R₁, R₁′) and (R₂, R₂′) being chosen independently of each other,
said labelled modified peptide being in the form of a racemate, any one of its enantiomers, or any one of the different tautomers corresponding to said racemates and enantiomers,
   provided that if a, b, c, d, e, f, g, h, i and j are equal to 0,
if (R₁, R₁′)═(—CH(CH₃)₂;H) then (R₂, R₂′) is different from (—CH(CH₃)—CH₂—CH₃;H), and

if (R₂, R₂′)═(—CH(CH₃)—CH₂—CH₃;H) then (R₁, R₁′) is different from (—CH(CH₃)₂;H) and provided that if said modified peptide is labelled with an iodine ¹²⁵I atom, if a, b, c, d, e, f, g, h, i and j are equal to 0 then (R₁, R₁′) is different from (—(CH₂)₃—CH₃;H).

In other words, the peptide of formula (Ia) below:

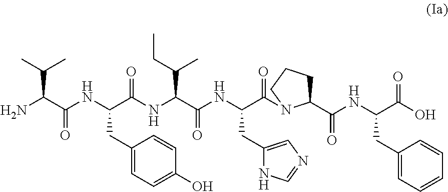

is excluded from the use according to the invention, irrespective of its labelling.

The peptide of formula (Ib) below:

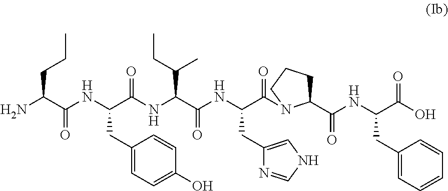

labelled with at least one iodine 125 atom (¹²⁵I), in particular labelled with an iodine 125 atom on the aromatic ring of the 4-hydroxy benzyl residue is also excluded from the use according to the invention.

Thus, the following two peptides of formula Ib1 and Ib2 are excluded:

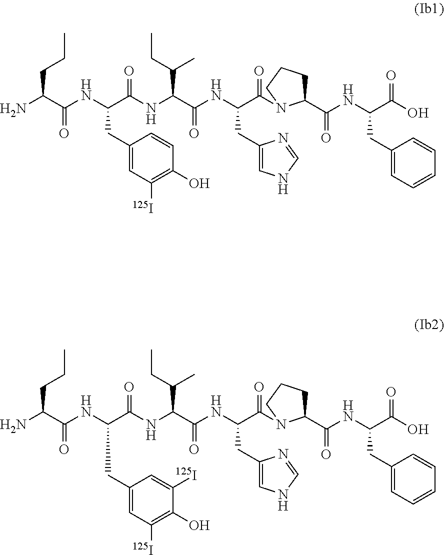

In an advantageous embodiment, the invention relates to a method of assay defined previously, in which the labelled modified peptide is chosen from the peptides of the following formulae:

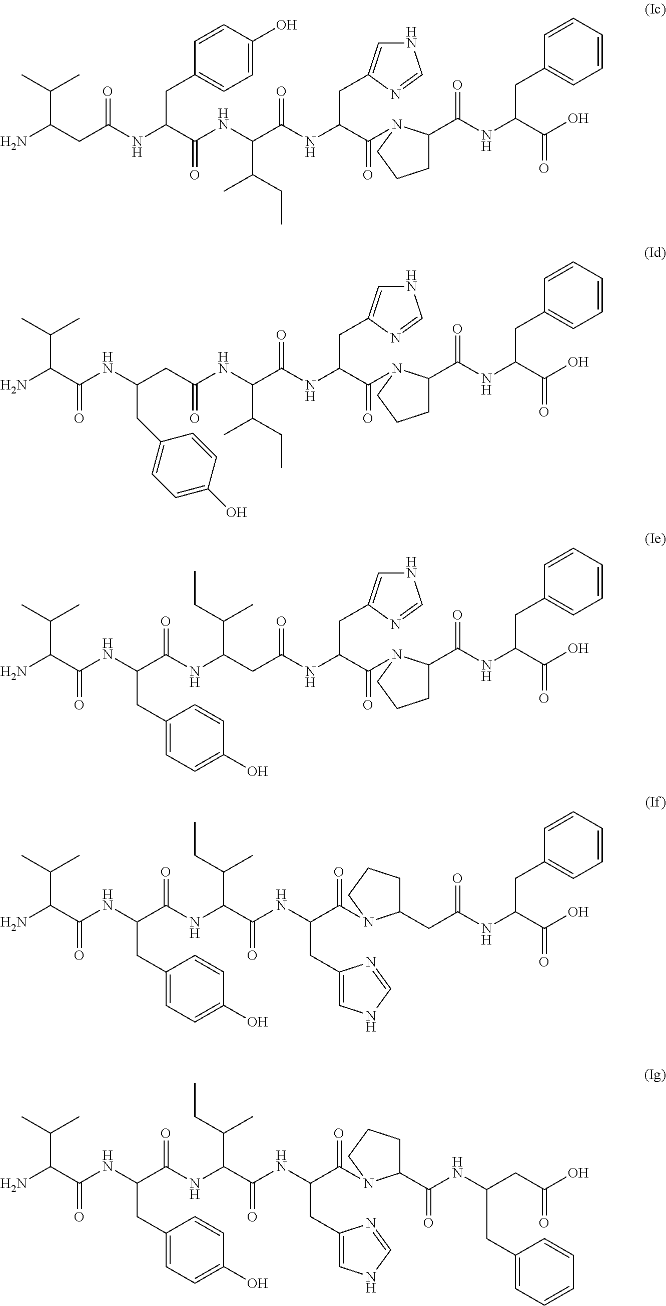

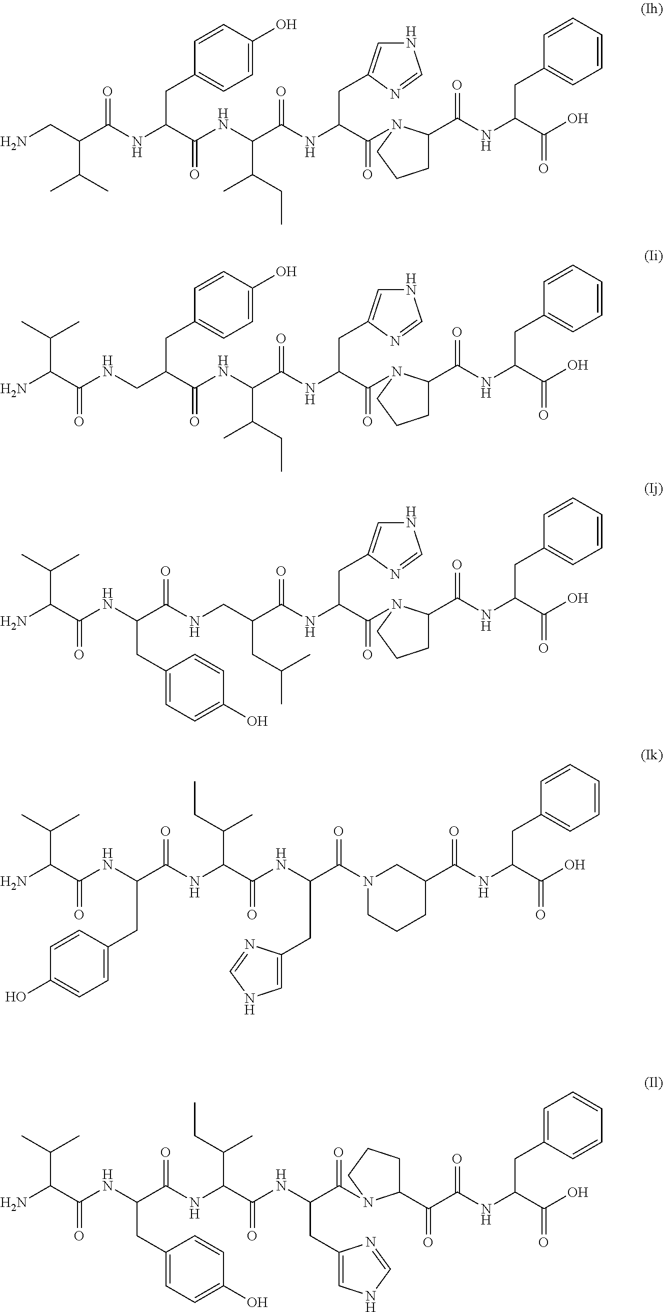

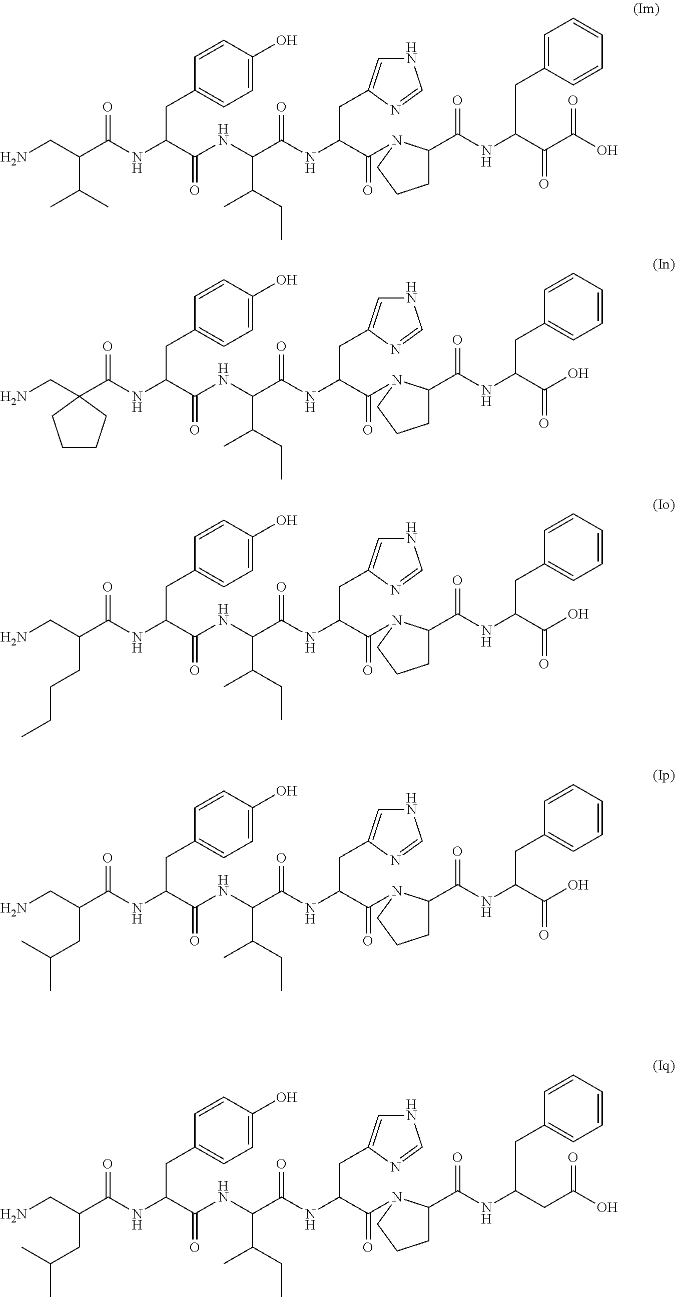

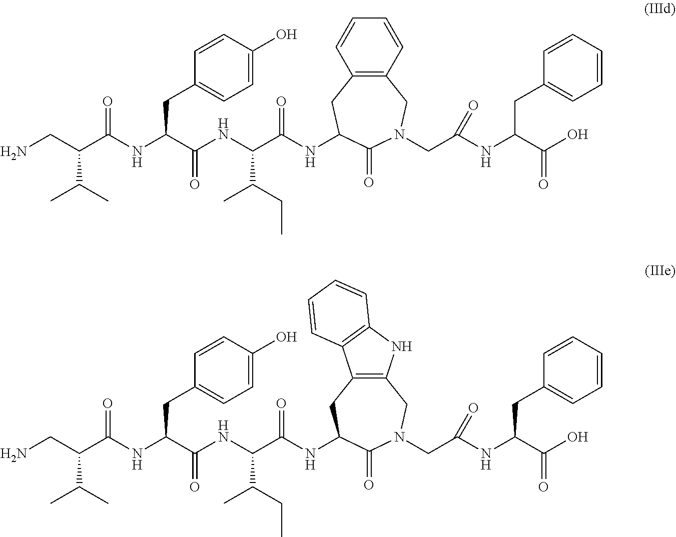

The invention also relates to a method of assay as defined above, where said labelled and modified peptide consists of the following sequence:

| | |
|---|---|
| X1-X2-X3-His-X4-X5 | (SEQ ID NO 22) |

where
X1 can be a Valine (Val), a Leucine (Leu), an Isoleucine (Ile), a Norleucine (Nle), a Cycloleucine (Cle) or a tert-leucine (Tle), or a β² or β³ derivative of one of these amino acids,
X2 can be a tyrosine, or a β² or β³ derivative of tyrosine,
X3 can be a Leucine (Leu), an Isoleucine (Ile), a Norleucine (Nle), a Cycloleucine (Cle) or a tert-leucine (Tle), or a β² or β³ derivative of one of these amino acids,
X4 can be a proline or a β² or β³ derivative of proline,
X5 can be a phenylalanine, or a β² or β³ derivative of phenylalanine,
and (His-X4) which can be

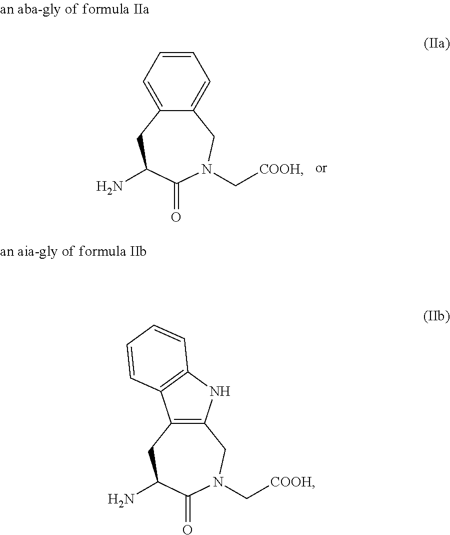

at least one of the amino acids X1 to X5 is a non-natural amino acid, and/or a natural or non-natural β-homo amino acid,
provided that the sequence SEQ ID NO 22 labelled with an iodine ¹²⁵I atom is different from the sequence Nle-Tyr-Ile-His-Pro-Phe (SEQ ID NO 23).

In another advantageous embodiment, the invention relates to a method for the abovementioned assay, where said labelled and modified peptide consists of the following sequence:

(SEQ ID NO 22)
X1-X2-X3-His-X4-X5

where
X1 can be a Valine (Val), a Leucine (Leu), an Isoleucine (Ile), a Norleucine (Nle), a Cycloleucine (Cle) or a tert-leucine (Tle), or a β² or β³ derivative of one of these amino acids,
X2 can be a tyrosine, or a β² or β³ derivative of tyrosine,
X3 can be a Leucine (Leu), an Isoleucine (Ile), a Norleucine (Nle), a Cycloleucine (Cle) or a tert-leucine (Tle), or a β² or β³ derivative of one of these amino acids,
X4 can be proline or a β² or β³ derivative of proline,
X5 can be a phenylalanine, or a β² or β³ derivative of phenylalanine, at least one of the amino acids X1 to X5 being a non-natural amino acid, and/or a natural or non-natural β-homo amino acid.
   provided that the sequence SEQ ID NO 22 labelled with an iodine ¹²⁵I atom is different from the sequence Nle-Tyr-Ile-His-Pro-Phe (SEQ ID NO 23).

According to the invention, the labelled modified peptide constituted by the amino acid sequence SEQ ID NO 22 corresponds to the peptide of general formula (I) defined above, in which all the amino acids are in an L configuration according to the L/D nomenclature known to a person skilled in the art.

The name as well as the structural formula of the different natural or non-natural amino acids, and the β2 or β3 derivatives of said natural or non-natural amino acids are shown in Table 1 below. The N column indicates the nature of the corresponding amino acid: natural (O) or non-natural (N).

| Amino acid | N | β3 Derivative | β2 Derivative |
|---|---|---|---|
| 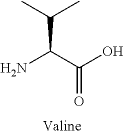 | O | 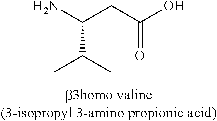 | 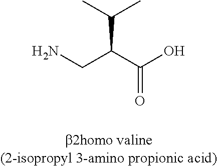 |
| 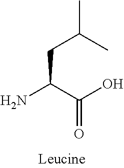 | O | 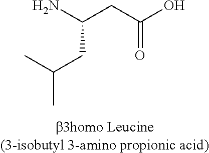 | 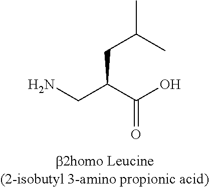 |
| 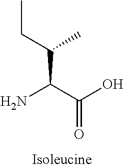 | O | 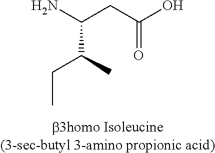 | 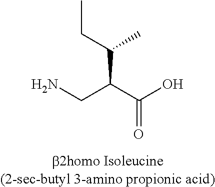 |
| 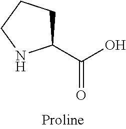 | O | 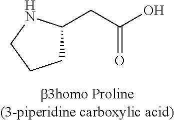 | 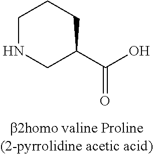 |
| 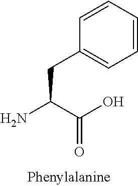 | O | 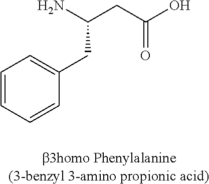 | 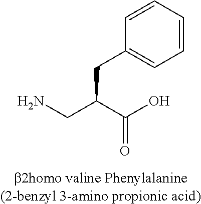 |
| 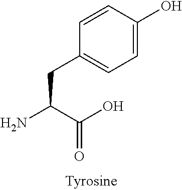 | O | 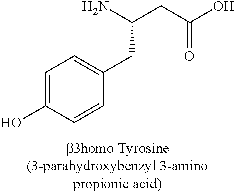 | 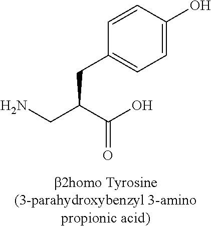 |
| 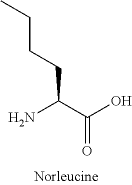 | N | 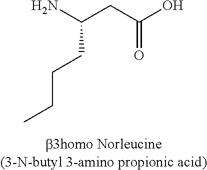 | 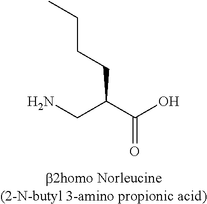 |
| 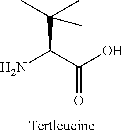 | N | 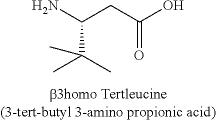 | 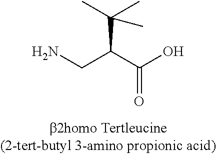 |
| 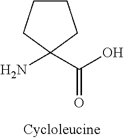 | N | 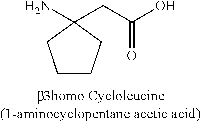 | 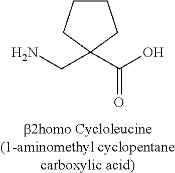 |

An advantageous embodiment of the invention relates to a method of assay defined previously, where said labelled and modified peptide consists of the following sequence:

(SEQ ID NO 22)
X1-X2-X3-His-X4-X5

and where at least one of the amino acids X1 to X5 is a natural or non-natural β-homo amino acid.

In other words, at least one of the amino acids X1, or X2, or X3, or X4 or X4 or X5 corresponds to an amino acid chosen from: β2homo valine, β3homo valine, β2homo leucine, 133homo leucine, β2homo isoleucine, β3homo isoleucine, β2homo tyrosine, β3homo tyrosine, β2homo proline, β3homo proline, β2homo phenylalanine, β3homo phenylalanine, β2homo norleucine, β3homo norleucine, β2homo tertleucine, β3homo tertleucine, β2homo cycloleucine and β3homo cycloleucine.

In another advantageous embodiment, the invention relates to a method of assay defined previously, where said labelled modified peptide is labelled
with at least one iodine ¹²⁵I atom on the tyrosine X2, preferentially a single iodine ¹²⁵I atom, in particular on the phenyl group, or
by the replacement of at least one of the hydrogen atoms with a tritium ³H atom.

In the case where the labelled modified peptide of the invention is labelled with an iodine 125 atom, said peptide possesses at least one ¹²⁵I atom, or two ¹²⁵I atoms, in the meta position on the benzyl ring of the tyrosine, or in the meta position on the benzyl ring of the β²homo tyrosine or β³homo tyrosine derivatives.

Preferentially, the peptide is labelled with a single iodine ¹²⁵I atom.

Another advantageous embodiment of the invention relates to a method of assay defined previously, where said labelled modified peptide is chosen from the following peptides:

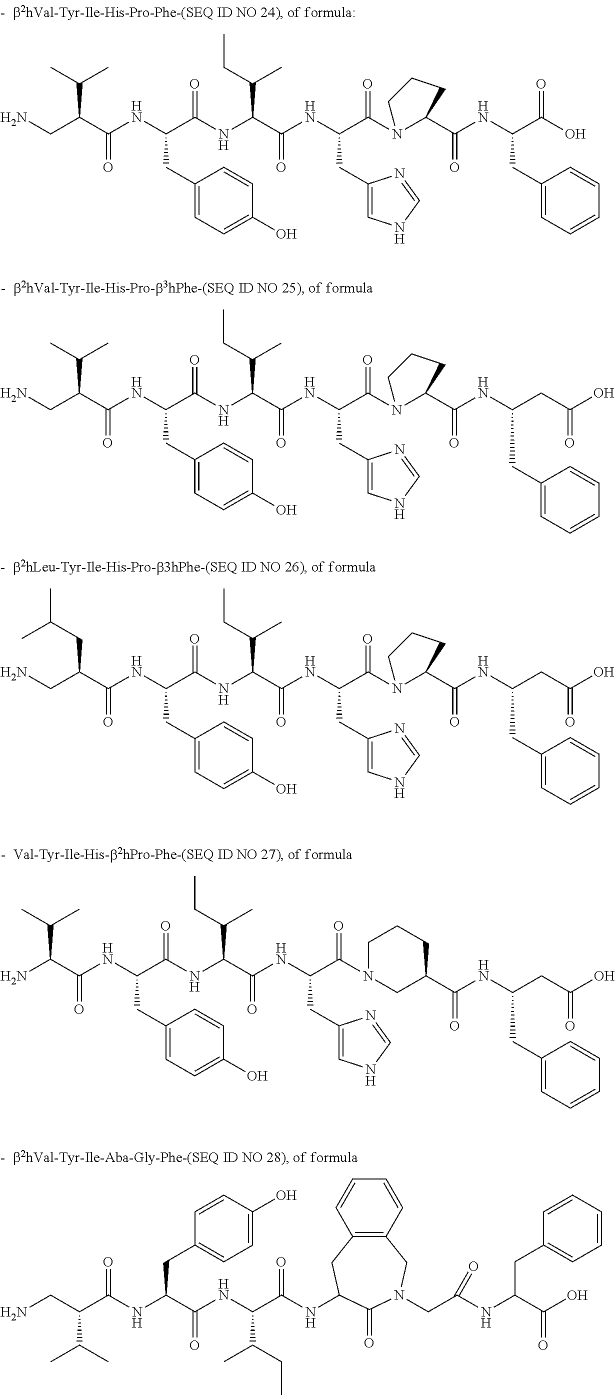

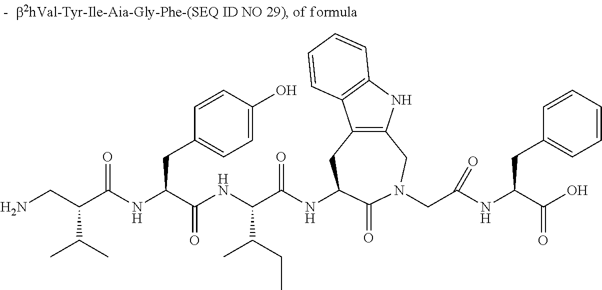

preferentially the peptide SEQ ID NO 25, said labelled modified peptide being iodinated. The above peptides have a high affinity for the circulating extracellular portion of IRAP, are stable, and can be easily detected by counting the radioactivity emitted by the iodine ¹²⁵I. Advantageously, the invention relates to a method of assay defined previously, where said labelled modified peptide is chosen from the following peptides:

(SEQ ID NO 24)
β2hVal-Tyr-Ile-His-Pro-Phe-,
(SEQ ID NO 25)
β2hVal-Tyr-Ile-His-Pro-β3hPhe-,
(SEQ ID NO 26)
β2hLeu-Tyr-Ile-His-Pro-β3hPhe-,
(SEQ ID NO 27)
Val-Tyr-Ile-His-β²hPro-Phe-,
(SEQ ID NO 28)
β²hVal-Tyr-Ile-Aba-Gly-Phe-,
and
(SEQ ID NO 29)
β²hVal-Tyr-Ile-Aia-Gly-Phe-,

preferentially the peptide SEQ ID NO 25,
said labelled modified peptide being tritiated.

The above peptides have a high affinity for the circulating extracellular portion of IRAP, are stable, and can be easily detected by counting the radioactivity emitted by the tritium ³H.

Another advantageous embodiment of the invention relates to a method of assay defined above, comprising
a stage of purification of said circulating extracellular portion of TRAP, and
a stage of quantification of said circulating extracellular portion purified in the previous stage, by means of at least one modified peptide of amino acid sequence SEQ ID NO 25 labelled with radioactive iodine ¹²⁵I, preferentially a single atom of radioactive iodine ¹²⁵I.

Another advantageous embodiment of the invention relates to a method of assay defined previously, comprising
a stage of purification of said circulating extracellular portion of IRAP, and
a stage of quantification of said circulating extracellular portion purified in the previous stage, by means of at least one tritiated modified peptide of amino acid sequence SEQ ID NO 25.

The invention also relates to a labelled modified peptide represented by general formula (III) below:

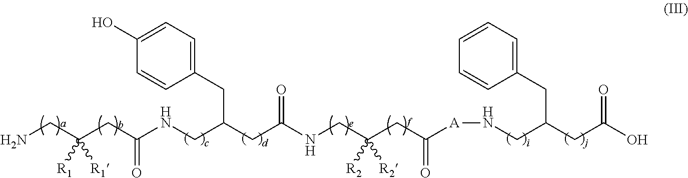

where a, b, c, d, e, f, g, h, i and j can be equal to 0 or 1, so that (a+b)≦1, (c+d)≦1, (e+f)≦1, (g+h)≦1 and (i,j)≦1, (a,b), (c,d), (e,f), (g,h) and (i,j) being independent of each other,
where (R₁, R₁′) are such that:
R1 is chosen from: a —CH(CH₃)₂ group, a —CH₂—CH(CH₃)₂ group, a —CH(CH₃)—CH₂—CH₃ group, a —(CH₂)₃—CH₃ group and a C(CH₃)₃ group, and R1′ is a hydrogen atom, or
R1 and R1′ form together with the carbon which carries them a cyclopentyl,
   where (R₂, R₂′) are such that:
if R2 is chosen from: a —CH₂—CH(CH₃)₂ group, a —CH(CH₃)—CH₂—CH₃ group, a —(CH₂)₃—CH₃ group and a C(CH₃)₃ group, R2′ is a hydrogen atom, or
R2 and R2′ form together with the carbon which carries them a cyclopentyl, the pairs (R₁, R₁′) and (R₂, R₂′) being chosen independently of each other,
   where A is chosen from the following groups

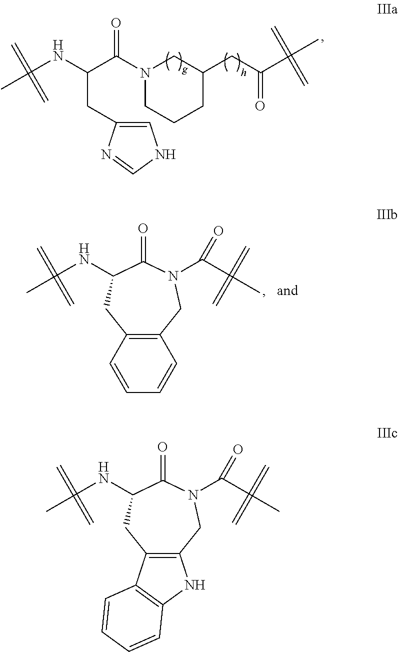

said labelled modified peptide being in the form of a racemate, any one of its enantiomers, or any one of the different tautomers corresponding to said racemates and enantiomers,
   provided that if A is represented by formula Ma and if a, b, c, d, e, f, g, h, i and j are equal to 0,
if (R₁, R₁′)═(—CH(CH₃)₂;H) then (R₂, R₂′) is different from (—CH(CH₃)—CH₂—CH₃;H), and
if (R₂, R₂′)═(—CH(CH₃)—CH₂—CH₃;H) then (R₁, R₁′) is different from (—CH(CH₃)₂;H) and provided that if said modified peptide is labelled with an iodine ¹²⁵I atom, if a, b, c, d, e, f, g, h, i and j are equal to 0 then (R₁, R₁′) is different from (—(CH₂)₃—CH₃;H).

The invention also relates to a labelled modified peptide represented by general formula (I) below:

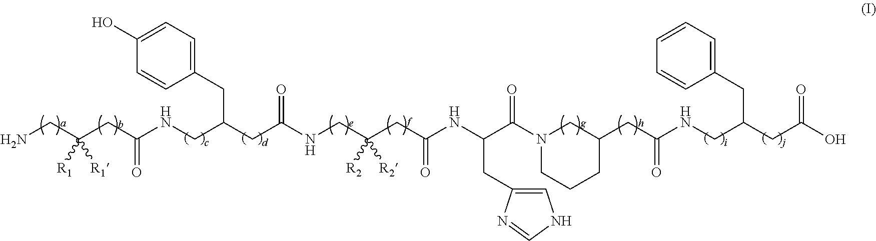

said peptide optionally modified by the presence of at least one non-natural amino acid, and/or at least one natural or non-natural β-homo amino acid,
   where a, b, c, d, e, f, g, h, i and j can be equal to 0 or 1, so that (a+b)≦1, (c+d)≦1, (e+f)≦1, (g+h)≦1 and (i,j)≦1, (a,b), (c,d), (e,f), (g,h) and (i,j) being independent of each other,
   where (R₁, R₁′) are such that:
R1 is chosen from: a —CH(CH₃)₂ group, a —CH₂—CH(CH₃)₂ group, a —CH(CH₃)—CH₂—CH₃ group, a —(CH₂)₃—CH₃ group and a C(CH₃)₃ group, and R1′ is a hydrogen atom, or
R1 and R1′ form together with the carbon which carries them a cyclopentyl,
   where (R₂, R₂′) are such that:
if R2 is chosen from: a —CH₂—CH(CH₃)₂ group, a —CH(CH₃)—CH₂—CH₃ group, a —(CH₂)₃—CH₃ group and a C(CH₃)₃ group, R2′ is a hydrogen atom, or
R2 and R2′ form together with the carbon which carries them a cyclopentyl, the pairs (R₁, R₁′) and (R₂, R₂′) being chosen independently of each other,
said labelled peptide being in the form of a racemate, any one of its enantiomers, or any one of the different tautomers corresponding to said racemates and enantiomers,
   provided that if a, b, c, d, e, f, g, h, i and j are equal to 0,
if (R1,R1′)═(—CH(CH₃)₂;H) then (R₂, R₂′) is different from (—CH(CH₃)—CH₂—CH₃;H) and if (R₂, R₂′)═(—CH(CH₃)—CH₂—CH₃;H) then (R1,R1′) is different from (—CH(CH₃)₂;H)
   and provided that if said labelled peptide is labelled with an iodine ¹²⁵I atom, if a, b, c, d, e, f, g, h, i and j are equal to 0 then (R1,R1′) is different from (—(CH₂)₃—CH₃;H).

The preceding peptides are novel.

An advantageous embodiment of the invention relates to a labelled peptide as defined previously, represented by the general formula Ic, Id, Ie, If, Ig, Ih, Ii, Ij, Ik, Il, Im, In, Io, IIId or IIIe as defined above.

In an advantageous embodiment the invention also relates to a modified and labelled peptide which consists of the following sequence:

| | |
|---|---|
| X1-X2-X3-His-X4-X5 | (SEQ ID NO 22) |

where
X1 can be a Valine (Val), a Leucine (Leu), an Isoleucine (Ile), a Norleucine (Nle), a Cycloleucine (Cle) or a tert-leucine (Tle), or a β² or β³ derivative of one of these amino acids,
X2 can be a tyrosine, or a β² or β³ derivative of tyrosine,
X3 can be a Leucine (Leu), an Isoleucine (Ile), a Norleucine (Nle), a Cycloleucine (Cle) or a tert-leucine (Tle), or a β² or β³ derivative of one of these amino acids,
X4 can be a proline or a β² or β³ derivative of proline,
X5 can be a phenylalanine, or a β² or β³ derivative of phenylalanine,
and (His-X4) which can be an aba-gly of formula IIa

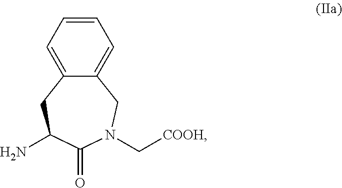

or an aia-gly of formula IIb

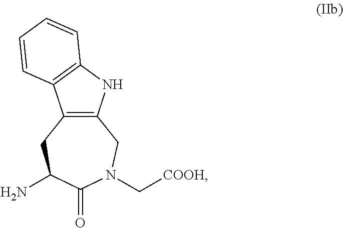

at least one of the amino acids X1 to X5 is a non-natural amino acid, and/or a natural or non-natural β-homo amino acid,
provided that the sequence SEQ ID NO 22 labelled with an iodine ¹²⁵I atom is different from the sequence Nle-Tyr-Ile-His-Pro-Phe (SEQ ID NO 23).

In another aspect, the invention relates to the abovementioned modified unlabelled peptides, in particular the peptides of formulae IIIb1 and IIIc1 below:

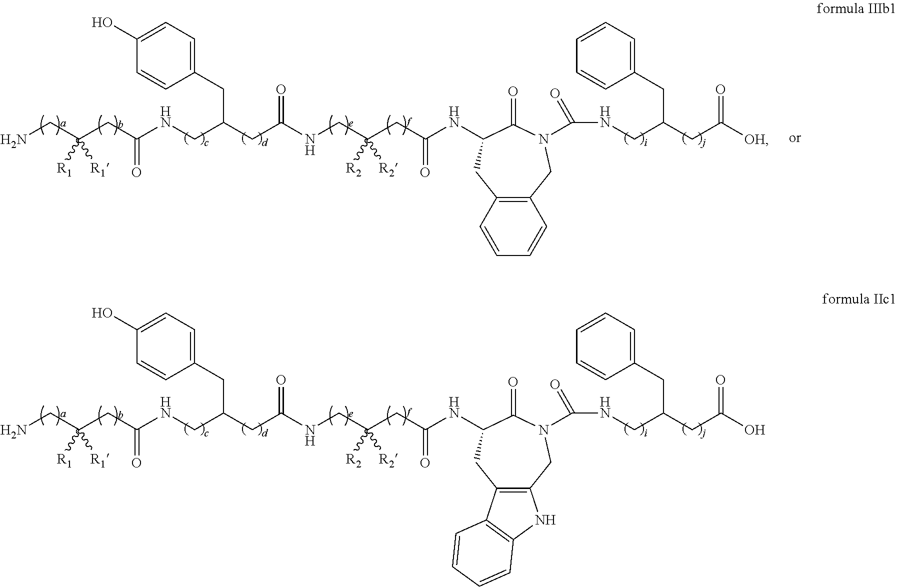

An advantageous aspect of the invention relates to a labelled peptide defined above, said peptide being labelled with at least one radioactive isotope consisting of the following sequence:

| | |
|---|---|
| X1-X2-X3-His-X4-X5, | (SEQ ID NO 22) |

said optionally modified peptide,
where
X1 can be a Valine (Val), a Leucine (Leu), an Isoleucine (Ile), or a non-natural amino acid derived from leucine, in particular Norleucine (Nle), Cycloleucine (Cle) or tert-leucine (Tle), or a β² or β³ derivative of one of these natural or non-natural amino acids,
X2 can be a tyrosine, or a β² or β³ derivative of tyrosine,
X3 can be a Leucine (Leu), an Isoleucine (Ile), or a non-natural amino acid derived from leucine, in particular Norleucine (Nle), Cycloleucine (Cle) or tert-leucine (Tle), or a β² or β³ derivative of one of these natural or non-natural amino acids,
X4 can be proline or a β² or β³ derivative of proline,
X5 can be a phenylalanine, or a β² or β³ derivative of phenylalanine, and
provided that said unmodified labelled peptide is different from angiotensin IV, and in particular that said unmodified labelled peptide is different from the sequence SEQ ID NO 21 (Val-Tyr-Ile-His-Pro-Phe),
provided that the sequence SEQ ID NO 22 labelled with an iodine ¹²⁵I atom is different from the sequence Nle-Tyr-Ile-His-Pro-Phe (SEQ ID NO 23).

Advantageously, the invention relates to a labelled modified peptide defined above, consisting of the following sequence:

| | |
|---|---|
| X1-X2-X3-His-X4-X5, | (SEQ ID NO 22) |

said peptide being modified so that at least one of the amino acids X1 to X5 is a natural or non-natural β-homo amino acid.

Another advantageous aspect of the invention relates to a labelled modified peptide defined previously, said labelled modified peptide being labelled:
with at least one iodine ¹²⁵I atom on the tyrosine X2, in particular on the phenyl group, or
by the replacement of at least one of the hydrogen atoms with a tritium ³H atom.

In another advantageous aspect, the invention relates to a labelled modified peptide mentioned previously, where said labelled modified peptide is chosen from the following peptides:

| | |
|---|---|
| β2hVal-Tyr-Ile-His-Pro-Phe-, | (SEQ ID NO 24) |
| βhVal-Tyr-Ile-His-Pro-β3hPhe-, | (SEQ ID NO 25) |
| βhLeu-Tyr-Ile-His-Pro-β3hPhe-, | (SEQ ID NO 26) |
| Val-Tyr-Ile-His-β²hPro-Phe-, | (SEQ ID NO 27) |
| β²hVal-Tyr-Ile-Aba-Gly-Phe-, | (SEQ ID NO 28) |
| and | |
| β²hVal-Tyr-Ile-Aia-Gly-Phe-, | (SEQ ID NO 29) |

preferentially the peptide SEQ ID NO 25,
said labelled modified peptide being iodinated, preferentially with a single iodine ¹²⁵I atom.

In another advantageous aspect, the invention relates to a labelled modified peptide mentioned previously, where said labelled modified peptide is chosen from the following peptides:

| | |
|---|---|
| β2hVal-Tyr-Ile-His-Pro-Phe-, | (SEQ ID NO 24) |
| β2hVal-Tyr-Ile-His-Pro-β3hPhe-, | (SEQ ID NO 25) |
| β2hLeu-Tyr-Ile-His-Pro-β3hPhe-, | (SEQ ID NO 26) |
| Val-Tyr-Ile-His-β²hPro-Phe-, | (SEQ ID NO 27) |
| β²hVal-Tyr-Ile-Aba-Gly-Phe-, | (SEQ ID NO 28) |
| and | |
| β²hVal-Tyr-Ile-Aia-Gly-Phe-, | (SEQ ID NO 29) |

preferentially the peptide SEQ ID NO 25,
said labelled modified peptide being tritiated.

The invention is illustrated by FIGS. 1A-Q, FIGS. 2A-Q and the following examples, without however being limited to said examples.

### FIGURE CAPTIONS

FIGS. 1A-Q represent the absorption spectra at 215 nm of the reversed-phase HPLC elutions in the presence of water/acetonitrile solvent, the mobile phase containing 0.1% TFA. The standard gradient corresponds to a migration over 20 min from 3% to 97% acetonitrile, at a rate of 1 mL/min.

FIG. 1A represents the UV absorption spectrum as a function of time of the peptide H-β3hVal-Tyr-Ile-His-Pro-Phe-OH.

FIG. 1B represents the UV absorption spectrum as a function of time of the peptide H-Val-β3hTyr-Ile-His-Pro-Phe-OH.

FIG. 1C represents the UV absorption spectrum as a function of time of the peptide H-Val-Tyr-β3hIle-His-Pro-Phe-OH.

FIG. 1D represents the UV absorption spectrum as a function of time of the peptide H-Val-Tyr-Ile-His-β3hPro-Phe-OH.

FIG. 1E represents the UV absorption spectrum as a function of time of the peptide H-Val-Tyr-Ile-His-Pro-β3hPhe-OH.

FIG. 1F represents the UV absorption spectrum as a function of time of the first diastereoisomer of the peptide H-β2hVal-Tyr-Ile-His-Pro-Phe-OH.

FIG. 1G represents the UV absorption spectrum as a function of time of the peptide of the second diastereoisomer of the peptide H-β2hVal-Tyr-Ile-His-Pro-Phe-OH.

FIG. 1H represents the UV absorption spectrum as a function of time of the mixture of the racemates of the peptide H-Val-β2hTyr-Ile-His-Pro-Phe-OH

FIG. 1I represents the UV absorption spectrum as a function of time of the first diastereoisomer of the peptide H-Val-Tyr-β2hLeu-His-Pro-Phe-OH

FIG. 1J represents the UV absorption spectrum as a function of time of the mixture of the racemates of the peptide H-Val-Tyr-β2hLeu-His-Pro-Phe-OH

FIG. 1K represents the UV absorption spectrum as a function of time of the peptide H-Val-Tyr-Ile-His-β2hPro-Phe-OH

FIG. 1L represents the UV absorption spectrum as a function of time of the mixture of the racemates of the peptide H-Val-Tyr-Ile-His-Pro-β2hPhe-OH

FIG. 1M represents the UV absorption spectrum as a function of time of the peptide H-β2hVal-Tyr-Ile-His-Pro-β3hPhe-OH

FIG. 1N represents the UV absorption spectrum as a function of time of the peptide

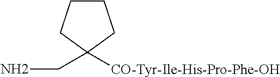

FIG. 1O represents the UV absorption spectrum as a function of time of the mixture of the racemates of the peptide H-β2hNle-Tyr-Ile-His-Pro-Phe-OH

FIG. 1P represents the UV absorption spectrum as a function of time of the mixture of the racemates of the peptide H-β2hLeu-Tyr-Ile-His-Pro-Phe-OH

FIG. 1Q represents the UV absorption spectrum as a function of time of the mixture of the racemates of the peptide H-β2hLeu-Tyr-Ile-His-Pro-β3hPhe-OH

FIGS. 2A-Q represent the absorption spectra at 215 nm of the reversed-phase HPLC elutions in the presence of water/methanol solvent, the mobile phase containing 0.1% TFA. The standard gradient corresponds to a migration over 20 min from 3% to 97% methanol, at a rate of 1 mL/min.

FIG. 2A represents the UV absorption spectrum as a function of time of the peptide H-β3hVal-Tyr-Ile-His-Pro-Phe-OH.

FIG. 2B represents the UV absorption spectrum as a function of time of the peptide H-Val-β3hTyr-Ile-His-Pro-Phe-OH.

FIG. 2C represents the UV absorption spectrum as a function of time of the peptide H-Val-Tyr-β3hIle-His-Pro-Phe-OH.

FIG. 2D represents the UV absorption spectrum as a function of time of the peptide H-Val-Tyr-Ile-His-β3hPro-Phe-OH.

FIG. 2E represents the UV absorption spectrum as a function of time of the peptide H-Val-Tyr-Ile-His-Pro-β3hPhe-OH.

FIG. 2F represents the UV absorption spectrum as a function of time of the first diastereoisomer of the peptide H-β2hVal-Tyr-Ile-His-Pro-Phe-OH.

FIG. 2G represents the UV absorption spectrum as a function of time of the peptide of the second diastereoisomer of the peptide H-β2hVal-Tyr-Ile-His-Pro-Phe-OH.

FIG. 2H represents the UV absorption spectrum as a function of time of the mixture of the racemates of the peptide H-Val-β2hTyr-Ile-His-Pro-Phe-OH

FIG. 2I represents the UV absorption spectrum as a function of time of the first diastereoisomer of the peptide H-Val-Tyr-β2hLeu-His-Pro-Phe-OH

FIG. 2J represents the UV absorption spectrum as a function of time of the mixture of the racemates of the peptide H-Val-Tyr-β2hLeu-His-Pro-Phe-OH

FIG. 2K represents the UV absorption spectrum as a function of time of the peptide H-Val-Tyr-Ile-His-β2hPro-Phe-OH

FIG. 2L represents the UV absorption spectrum as a function of time of the mixture of the racemates of the peptide H-Val-Tyr-Ile-His-Pro-β2hPhe-OH

FIG. 2M represents the UV absorption spectrum as a function of time of the peptide H-β2hVal-Tyr-Ile-His-Pro-β3hPhe-OH

FIG. 2N represents the UV absorption spectrum as a function of time of the peptide

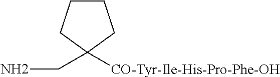

FIG. 2O represents the UV absorption spectrum as a function of time of the mixture of the racemates of the peptide H-β2hNle-Tyr-Ile-His-Pro-Phe-OH

FIG. 2P represents the UV absorption spectrum as a function of time of the mixture of the racemates of the peptide H-β2hLeu-Tyr-Ile-His-Pro-Phe-OH

FIG. 2Q represents the UV absorption spectrum as a function of time of the mixture of the racemates of the peptide H-β2hLeu-Tyr-Ile-His-Pro-β3hPhe-OH

FIGS. 3A-D represent HPLC profiles of iodination of the peptides Nos. 6, 11, and 17 and angiotensin IV. The X-axis represents the retention time in minutes, and the Y-axis represents the intensity expressed in mV/I*.

FIG. 3A represents the profile of the peptide of angiotensin IV,

FIG. 3B represents the profile of the ligand 6,

FIG. 3C represents the profile of the ligand 11, and

FIG. 3D represents the profile of the ligand 16.

FIG. 4 represents HPLC profiles of iodination of the mono-iodinated peptide No. 17. The X-axis represents the retention time in minutes, and the Y-axis represents the intensity expressed in mV/I*.

FIG. 5 represents the binding of angiotensin IV and peptide derivatives (ligands 6, 11 and 17) to the recombinant IRAP-His protein. The X-axis represents the quantity of recombinant IRAP expressed in μg/mL, the Y-axis represents the radioactivity of iodine 125 expressed in B/Bmax.

### EXAMPLES

### Example 1

### Synthesis of the Peptides

The synthesis of all the peptides was carried out by solid phase peptide synthesis using amino acids protected in the terminal amino position by tert-butoxycarbonyl (Boc) or N-9-fluorenylmethoxycarbonyl (Fmoc).

The peptides were synthesized on a Merrifield Boc-Phe resin (0.57 mmol/g), a Wang Fmoc-Phe resin (0.76 mmol/g) or a 2-chlorotrityl chloride resin (1.5 mmol/g).

The protective groups of the side chains were Tyr(t-Bu), β3-hTyr (t-Bu), and His(Trt) for the Fmoc synthesis, and Tyr (2,6-di-Cl-Bzl) and His(Tos) for the Boc synthesis.

The Fmoc protective groups were washed out with a 20% solution of piperidine in dimethyl formamide (DMF) (2×5 min), and the cleavage of the Boc protective groups was carried out in a 50% solution of trifluoroacetic acid (TFA) in dichloromethane (DCM) (2×10 min) and a 10% solution of triethylamine (TEA) in DCM (2×5 min) was used for the neutralization. The coupling of the amino acids was carried out in DMF/DCM (1v/1v) using O-(Benzotriazol-1-yl)-N,N,N′,N′-tetramethyluronium tetrafluoroborate (TBTU) (3 equivalents) and diisopropylethylamine (DIPEA) (6 equivalents).

The synthesis protocols were the following:

### Standard Fmoc Cycle:

| Stage | Process | Repetition | Time | Remark |
|---|---|---|---|---|
| 1 | Stabilization of the resin | | 1 h | |
| | with CH₂Cl₂ | | | |
| 2 | Fmoc deprotection | 2x | 5 and 10 min | |
| | 20% piperidine/DMF | | | |
| 3 | Washing with CH₂Cl₂ | 3x | 1 min | |
| 4 | Washing with iPrOH | 3x | 1 min | |
| 5 | Washing with CH₂Cl₂ | 3x | 1 min | |
| 6 | Colorimetric test | | | If negative |
| | | | | return to |
| | | | | stage 3 |
| 7 | Coupling | | 2 h or | |
| | | | overnight | |
| 8 | Washing with CH₂Cl₂ | 3x | 1 min | |
| 9 | Washing with iPrOH | 3x | 1 min | |
| 10 | Washing with CH₂Cl₂ | 3x | 1 min | |
| 11 | Colorimetric test | | | If positive |
| | | | | return to |
| | | | | stage 7 |
| 12 | Repetition of stages | | | |
| | 2 → 11 | | | |

The 3 eq of amino acids (with respect to the resin) and the 6 eq of DIPEA (with respect to the resin) were dissolved in CH₂Cl₂ (10 mL for 1 g of resin), containing, if necessary, a small quantity of DMF to facilitate the solubility of the amino acid. The 2-chlorotritilchloride resin was prestabilized in CH₂Cl₂ for 1 h, and after the addition of the mixture the resin was mixed for 30-120 min. Later, the resin was washed three times with a CH2Cl2/MeOH/DIPEA mixture (17:2:1), then twice with DMF and three times with CH₂Cl₂.

Standard Boc cycle:

| Stage | Process | Repetition | Time | Remark |
|---|---|---|---|---|
| 1 | Stabilization of the | | 1 h | |
| | resin with Boc | | | |
| | deprotection | | | |
| 2 | 50% TFA/CH₂Cl₂ | 2x | 5 and 15 min | |
| 3 | Washing with CH₂Cl₂ | 3x | 1 min | |
| 4 | Washing with iPrOH | 3x | 1 min | |
| 5 | Washing with CH₂Cl₂ | 3x | 1 min | |
| 6 | Neutralisation | | | |
| | 10% DIPEA/CH₂Cl₂ | | | |
| 7 | Washing with CH₂Cl₂ | 3x | 1 min | |
| 8 | Washing with iPrOH | 3x | 1 min | |
| 9 | Washing with CH₂Cl₂ | 3x | 1 min | |
| 10 | Colorimetric test | | | If negative |
| | | | | return to |
| | | | | stage 3 |
| 11 | Coupling | | 2 h or | |
| | | | overnight | |
| 12 | Washing with CH₂Cl₂ | 3x | 1 min | |
| 13 | Washing with iPrOH | 3x | 1 min | |
| 14 | Washing with CH₂Cl₂ | 3x | 1 min | |
| 15 | Colorimetric test | | | If positive |
| | | | | return to |
| | | | | stage 7 |
| 16 | Repetition of stages 2 → 15 | | | |

The Boc-amino acid was dissolved in EtOH (2 mL/mmol) and water (0.5 mL/mmol), the pH is adjusted to 7 with a 2 M aqueous solution of Cs₂CO₃. The solution was evaporated by drying, the residues were evaporated twice with dioxane. The Merrifield resin was prestabilized in CH₂Cl₂ for 1 h and washed with DMF. The amino acid caesium salt (1.2 eq) in DMF was added to the resin, and the reaction medium was heated at 50° C. overnight. At the end of the reaction, the resin was washed three times in DMF, three times in a DMF/water mixture (1:1), three times in DMF, three times in CH₂Cl₂, and three times in MeOH.

In both strategies (Boc and Fmoc), 3 eq. of amino acid, 3 eq. of TBTU and 6 eq. of DIPEA were mixed and left for 3 min to allow activation. The solution is then added to the resin and stirred for two hours or overnight.

The colorimetric test corresponds to a Kaiser test. This test consists of 3 solutions:
A: 5 g of ninhydrin in 100 mL of n-BuOH;
B: 80 g of phenol in 20 mL of n-BuOH;
C: 2 mL of 0.001 M aqueous KCN in 98 mL of pyridine.

A few beads of resin are placed in a test tube. 3 drops of each of the solutions are added and the tube is heated at 100° C. for 5 min. When the resin or the solution is colourless or yellowish, the result is negative. A blue coloration indicates the presence of a primary amine, and a brown/red coloration indicates the presence of a secondary amine

The peptides were cleaved from the resin by treatment with a TFA/H₂O/Triethylsilane mixture (TES) (95:2.5:2.5) for 2 h, or with hydrofluoric acid HF, or a TFA/trifluoromethanesulphonic acid mixture (TFMSA)/TES (20:2:3) in the case of a Merrifield resin. In both cases ethanol is added in order to precipitate the peptides. The precipitates are washed with EtOH.

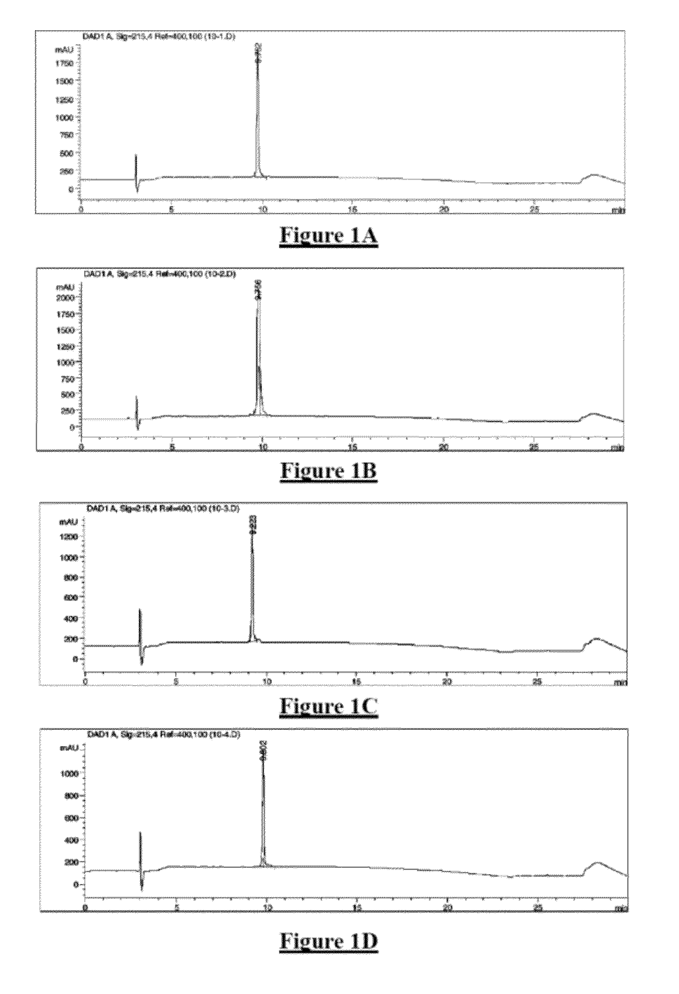

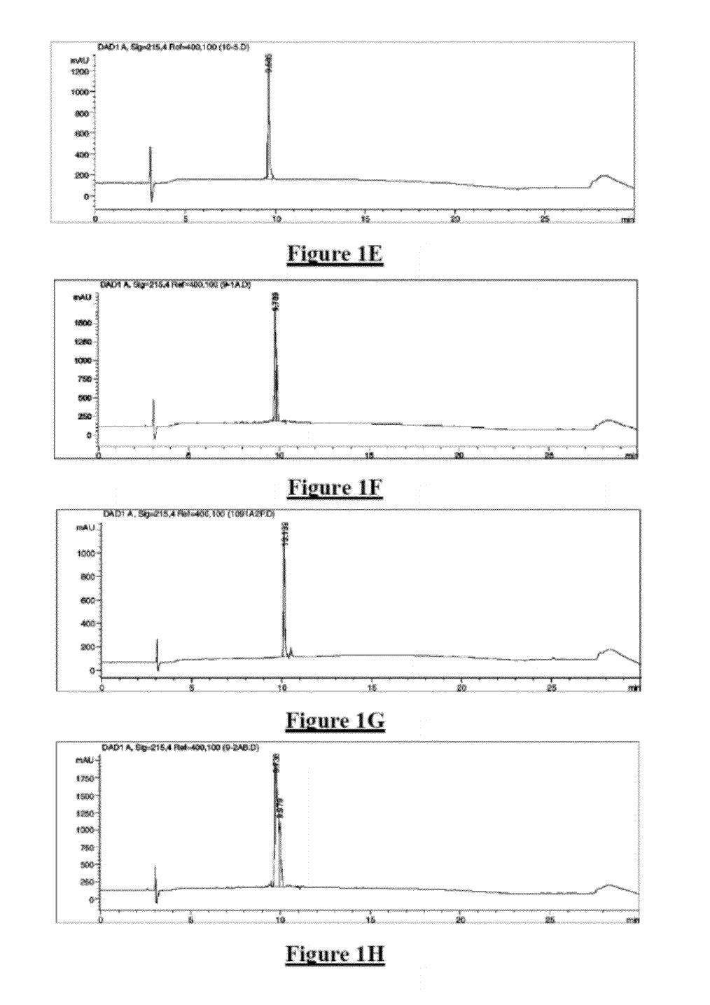

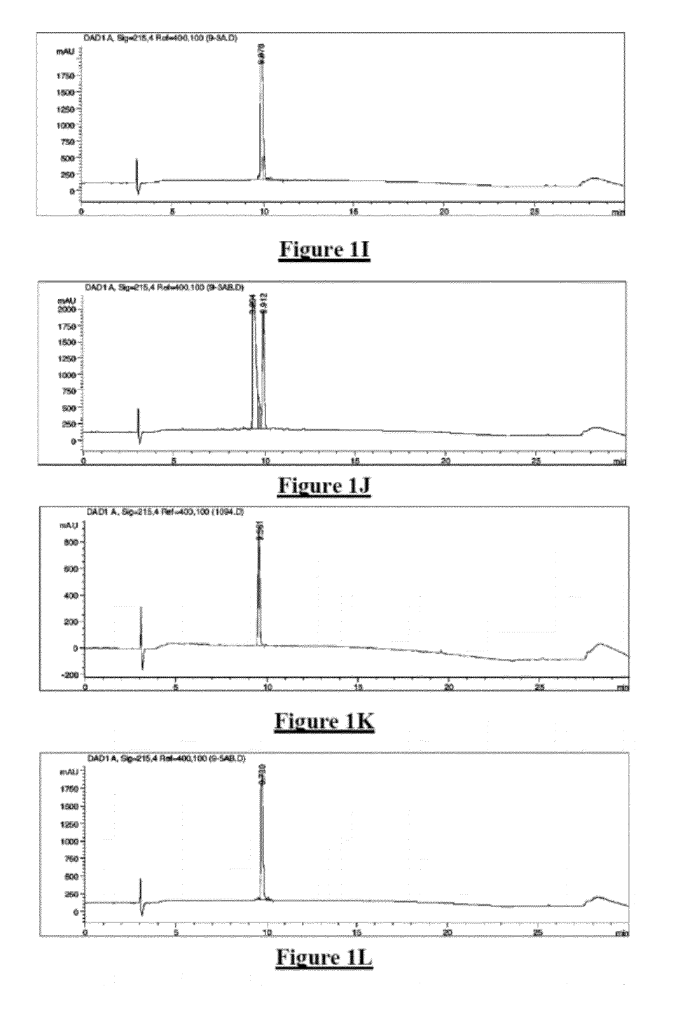

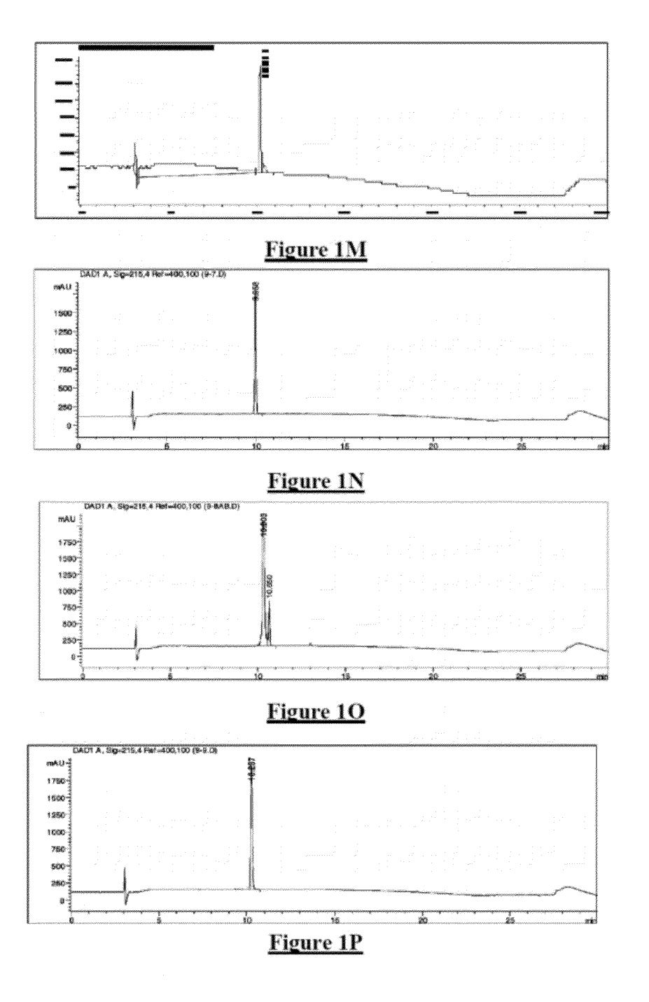

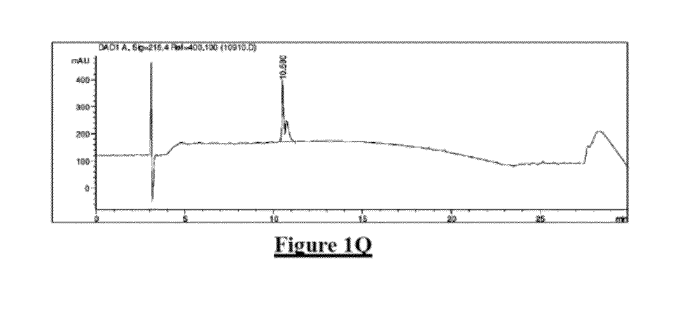

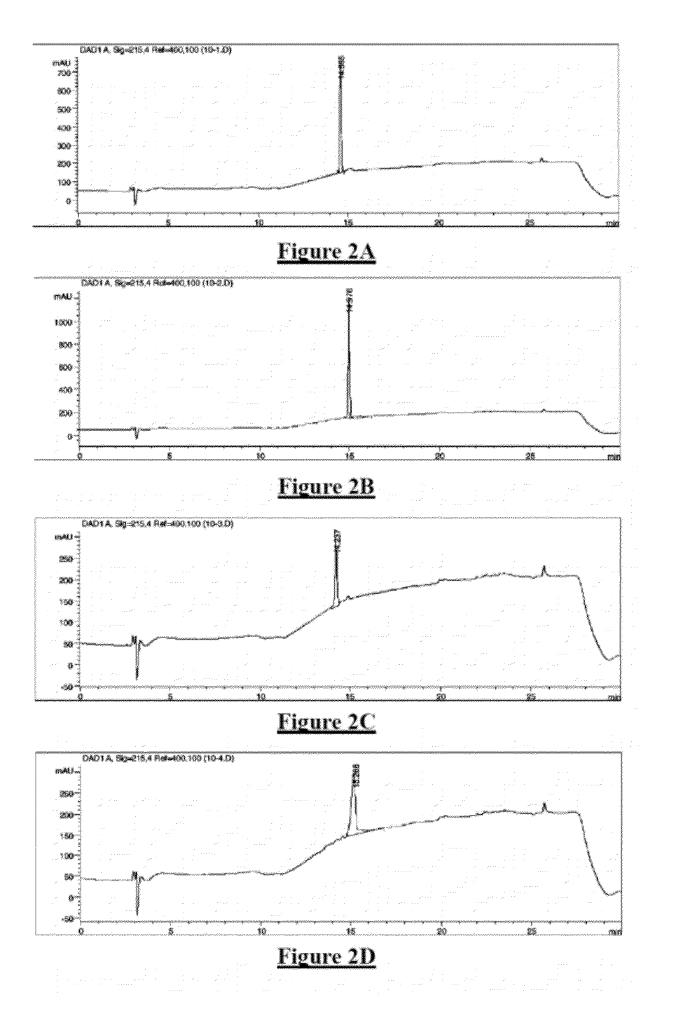

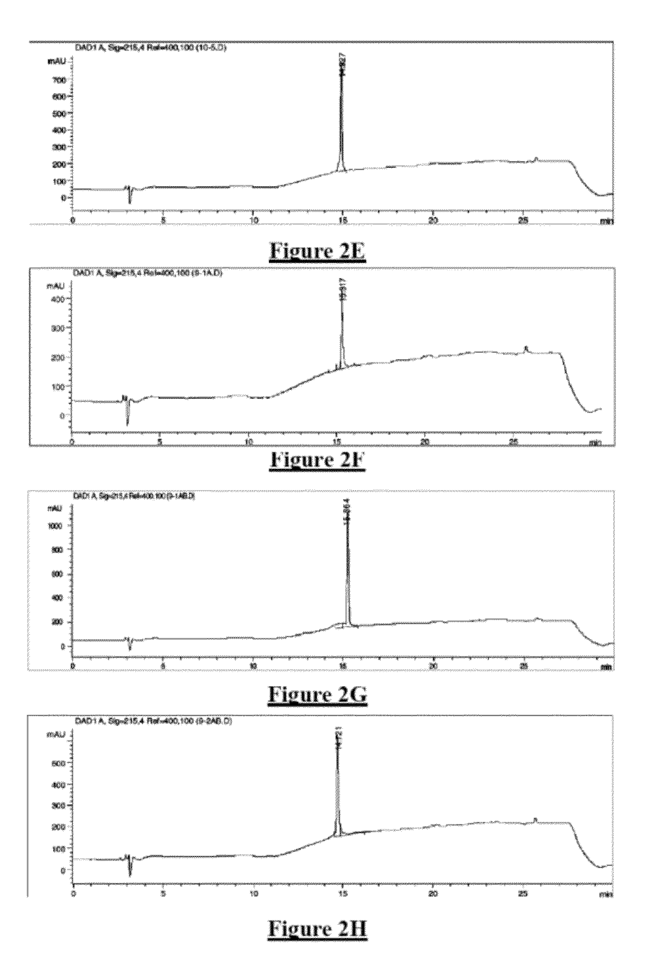

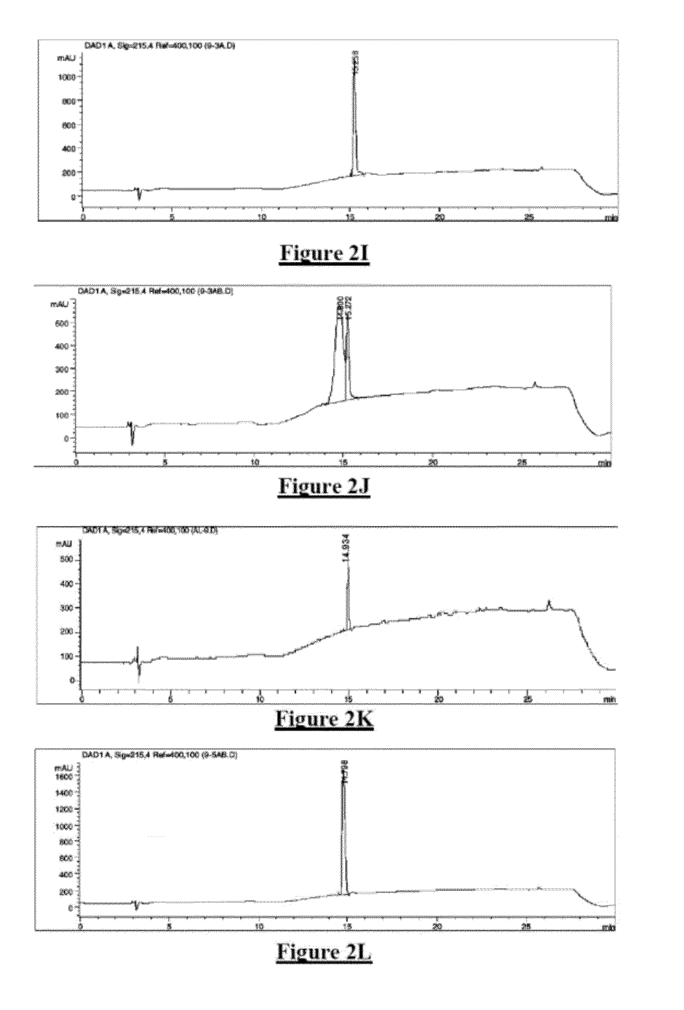

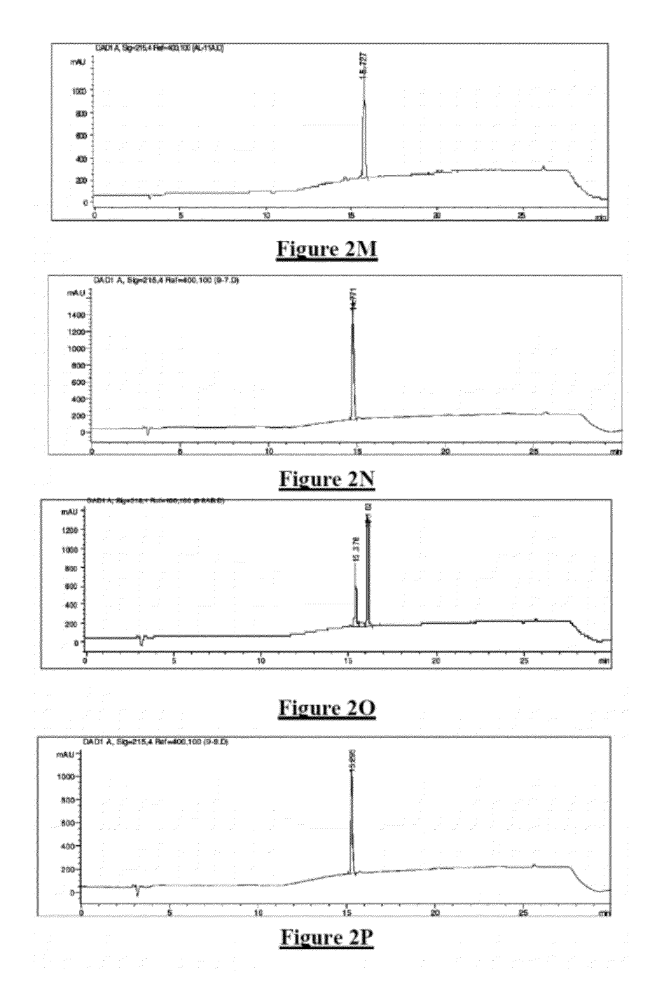

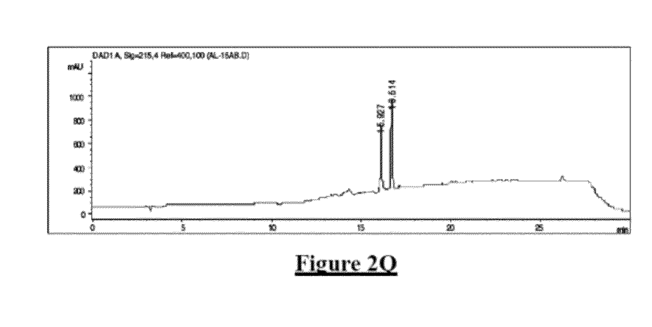

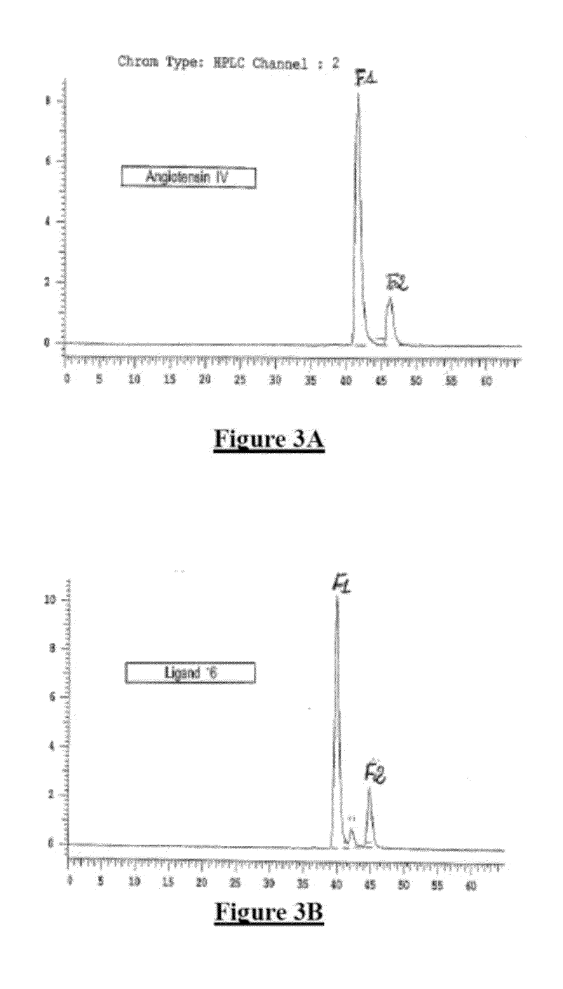

The peptides were purified by reversed-phase HPLC on a C18 Supelco Discovery BIO Wide Pore column. Each peptide was at least 98% pure after a thin layer chromatography test (TLC) and reversed-phase HPLC analysis in a water/acetonitrile solvent (FIGS. 1A-1Q), or in a water/methanol solvent (FIGS. 2A-2Q).

The molecular weights were confirmed by electrospray ionization-mass spectrometry (ESI-MS).

The following table summarizes the properties of the peptides synthesized, M calc corresponds to the predicted mass, ESI MS corresponds to the mass obtained by mass spectrometry, R % corresponds to the yield in percent with respect to the crude peptides, Time Ret1 corresponds to the retention time during the water/acetonitrile elution, Purity % 1 corresponds to the purity of the product obtained after the water/acetonitrile elution, Time Ret2 corresponds to the retention time during the water/methanol elution, Purity %2 corresponds to the purity of the product obtained after the water/methanol elution.

| | | | | Time | Purity | Time | Purity |
|---|---|---|---|---|---|---|---|
| Compound | M calc | ESI MS | R % | Ret1 | %1 | Ret2 | %2 |
| H-β3hVal-Tyr-Ile-His-Pro-Phe-OH | 788.42 | 789.41 | 46.6 | 9.75 | >99 | 14.59 | >99 |
| (AL-1) | | | | | | | |
| H-Val-β3hTyr-Ile-His-Pro-Phe-OH | 788.42 | 789.33 | 70 | 9.75 | >99 | 14.97 | >99 |
| (AL-2) | | | | | | | |
| H-Val-Tyr-β3hIle-His-Pro-Phe-OH | 788.42 | 789.42 | 41.6 | 9.22 | >99 | 14.23 | >99 |
| (AL-3) | | | | | | | |
| H-Val-Tyr-Ile-His-β3hPro-Phe-OH | 788.42 | 789.30 | 77.9 | 9.80 | >99 | 15.26 | >99 |
| (AL-4) | | | | | | | |
| H-Val-Tyr-Ile-His-Pro-β3hPhe-OH | 788.42 | 789.30 | 87.3 | 9.58 | >99 | 14.92 | >99 |
| (AL-5) | | | | | | | |
| H-β2hVal-Tyr-Ile-His-Pro-Phe-OH | 788.42 | 789.39 | 83.6 | 9.79 | >99 | 15.31 | >99 |
| (AL-6a) | | | | | | | |
| H-β2hVal-Tyr-Ile-His-Pro-Phe-OH | 788.42 | 789.39 | 83.6 | 10.13 | >99 | 15.86 | >99 |
| (AL-6b) | | | | | | | |
| H-Val-β2hTyr-Ile-His-Pro-Phe-OH | 788.42 | 789.42 | 86.8 | 9.73 | >99 | 14.72 | >99 |
| (AL-7ab) | | | | 9.97 | | | |
| H-Val-Tyr-β2hLeu-His-Pro-Phe-OH | 788.42 | 789.55 | 89 | 9.87 | >99 | 15.25 | >99 |
| (AL-8a) | | | | | | | |
| H-Val-Tyr-β2hLeu-His-Pro-Phe-OH | 788.42 | 789.55 | 89 | 9.29 | >99 | 14.80 | >99 |
| (AL-8ab) | | | | 9.91 | | 15.27 | |
| H-Val-Tyr-Ile-His-β2hPro-Phe-OH | 788.42 | 789.40 | 77.9 | 9.56 | >99 | 14.93 | >99 |
| (AL-9) | | | | | | | |
| H-Val-Tyr-Ile-His-Pro-β2hPhe-OH | 788.42 | 789.50 | 37.3 | 9.73 | >99 | 14.79 | >99 |
| (AL-10ab) | | | | | | | |
| H-β2hVal-Tyr-Ile-His-Pro-β3hPhe-OH | 802.44 | 803.37 | 53.3 | 10.16 | >99 | 15.72 | >99 |
| (AL-11) | | | | | | | |
| 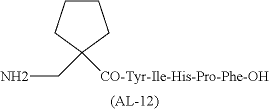 | 800.94 | 801.39 | 89 | 9.96 | >99 | 14.77 | >99 |
| H-β2hNle-Tyr-Ile-His-Pro-Phe-OH | 802.96 | 804.01 | 96 | 10.30 | >99 | 15.38 | >99 |
| (AL-13ab) | | | | 10.65 | | 16.10 | |
| H-β2hLeu-Tyr-Ile-His-Pro-Phe-OH | 802.96 | 804.00 | 97 | 10.24 | >99 | 15.29 | >99 |
| (AL-14ab) | | | | | | | |
| H-β2hLeu-Tyr-Ile-His-Pro-β3hPhe-OH | 816.99 | 817.45 | 65 | 10.5 | >99 | 15.92 | >99 |
| (AL-15ab) | | | | | | 16.51 | |

During the synthesis, all the β2 homo amino acids used were in the form of racemates, and after synthesis, two diastereoisomers were obtained. The separation of these diastereoisomers is indicated by the presence of the letters “a” and “b”. When the diastereoisomers could not be separated, they are represented by “ab”. By convention, the “a” diastereoisomers are eluted before the “b” diastereoisomers during reversed-phase HPLC.

### Example 2

### Labelling with Tritium

The neosynthesized and non-purified peptides were tritiated by catalytic saturation according to the procedure described in Tóth G et al. (1997 Methods Mol. Biol. vol 73, pp: 219-30).

The gaseous tritiated dihydrogen ³H₂ used is obtained from Technobexport, (Russia) and contains a quantity of tritium greater than or equal to 98%.

The radioactivity of the crude peptides was measured using a TRI-CARB 2100TR scintillation counter in a scintillant comprising a toluene-Triton X-100 mixture. The measurement of the radioactivity was approximately 100mCi (3.7 GBq)

The tritiated peptides were purified by HPLC using a Grace Vydac 218TP54 C18 column, and detection of the scintillation liquid was carried out on a Can berra Packard Radiomatic 505TR Flow Radiochromatography detector using Ultima-Flo M scintillant. The specific activity of the tritiated purified peptides was measured by HPLC using a standard curve. The specific activity obtained was comprised between approximately 30.0 Ci mmol⁻¹ and 45.0 Ci mmol⁻¹.

### Example 3

### Labelling with Iodine 125 (¹²⁵I)

The iodination of the peptides can be carried out according to several protocols known to a person skilled in the art. The principle of the iodination of proteins is based on the conversion of I⁻ (NaI) to I⁺ or I³⁻ in the presence of oxidizing agents such as chloramine-T, iodogen (1,3,4,6-tetrachloro-3α,6α-diphenyl-glycoluril), N-chloro-benzenesulphonamide or lactoperoxidase. I⁺ or I³⁻ are reactive species which attack the aromatic ring of the tyrosines in the meta position as described in “Antibodies: a laboratory manual” E. Harlow and D. Lane, Cold Spring Harbor Laboratory Press, 1988 pp. 324-339.

Material for the Iodination with N-Chloro-Benzenesulphonamide

The radioactive iodine ¹²⁵I is used in the form of Na¹²⁵I in a basic solution at approximately 1 mCi(37 MBq)/10 μl IODINE-125(Amersham; IMS30).

### Reagents

0.1 M phosphate buffer, pH 7.0
Tris-buffered saline (TBS) buffer
5% BSA in TBS
Stabilization solution (10% sodium thiosulphate+0.1N NaOH)
IODO-BEAD™ (PIERCE): beads grafted with an oxidizing agent (N-chloro-benzenesulphonamide)
Hamilton microsyringe (model 702, 25 μl, needle 22S, style 2)
desalting column (Bio-rad DG-10 etc.)

### Procedure

1. 200 μl, of 0.1M phosphate buffer, pH 7 is mixed with 500 μCi of Na¹²⁵I (5 μL) and 1 to two doses of IODO-BEADs in a plastic tube.
2. The mixture is incubated at ambient temperature for 5 min.
3. The peptides are added to the solution (between 1 and 100 ng of peptide)
4. The new mixture is incubated at ambient temperature for 10-25 min
5. The supernatant is removed and placed on a desalting column, in order to remove the free salts, Na¹²⁵I and r or I³⁻.
6. The fractions of the column are collected, and the radioactivity of each of the fractions is measured, using a scintillation counter.
7. In the radioactive fractions, ¼ of the volume of a 5% BSA solution is added in order to prevent decomposition by radiation.

### Alternative Iodination Procedure

### Reagents

Labelling buffer=250 mM ammonium acetate buffer pH 5.3
Conditioning buffer=PBS+0.1% BSA
Purification buffer=20% Acetonitrile+0.5% TFA
HPLC purification, C18 column
reconstitution of the ligands in labelling buffer [ligand]=100 μg/ml

| | ligands sequence | MW |
|---|---|---|
| 6 | H-β2hVal-Tyr-Lle-His-Pro-Phe-OH | 788.42 |
| 11 | H-Val-Tyr-Lle-His-β2hPro-Phe-OH | 788.42 |
| 17 | H-β2hLeu-Tyr-Lle-His-Pro- | 817.45 |
| | β3hPhe-OH | |
| Angiotensin | Val-Tyr-Ile-His-Pro-Phe | 774.91 |
| IV | | |

### Labelling

Specific activity=500 μCi/μg or 18.5 MBq/μg of ligand
5 μg (50mL) ligand
92.5 MBq of iodine
15 μg ChT
contact time=60 s
15 μg MBS
contact time=30 s
purification=reversed-phase HPLC OD monitoring at 275 nm following the gradient below:

| min | TFA | Acetonitrile |
|---|---|---|
| 0 | 100 | 0 |
| 2 | 90 | 10 |
| 10 | 80 | 20 |
| 60 | 60 | 40 |
| 70 | 0 | 100 |
| 80 | 0 | 100 |

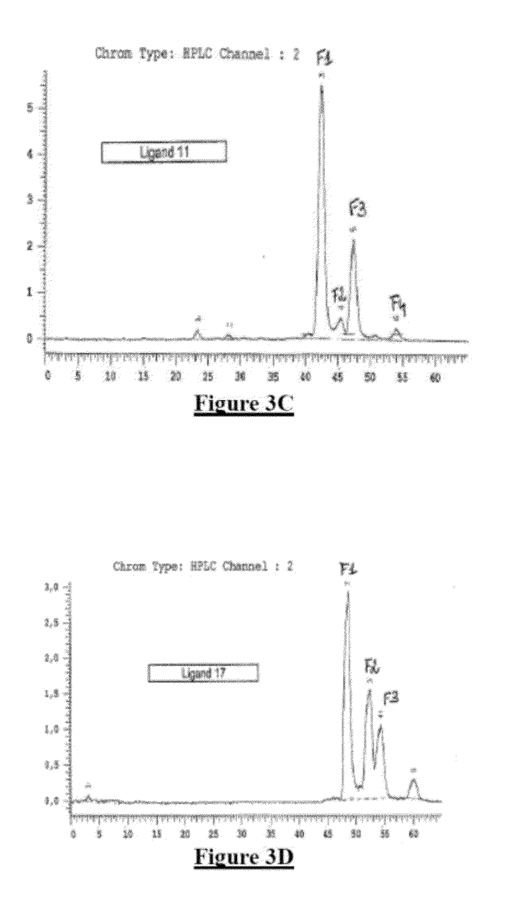

Results: the HPLC profiles of the synthetic TRAP ligands and of angiotensin IV labelled with iodine 125 are represented in FIGS. 3A to 3D

Determination of the “Mono-Iodinated” Fraction
1st stage=labelling of the ligand 17 with “cold” iodine, purification by HPLC, analysis of the fractions obtained by mass spectrometry, collection of the “mono-iodinated” fraction
2nd stage=simultaneous injection of the “mono-iodinated” fraction obtained during the 1st stage and the fraction F1 of the ligand 17 labelled with iodine 125
3rd stage=co-elution of fraction F1 of the ligand labelled with I125 and the “mono-iodinated” fraction

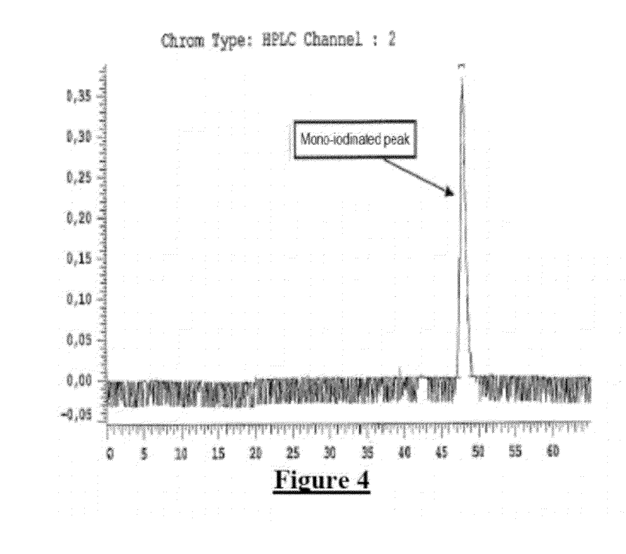

FIG. 4 shows that fraction F1 is indeed “mono-iodinated”.

### Example 4

### Specificity of the Peptides: Measurement of the Labelled Peptide/Extracellular Portion of IRAP Bond

Measurement of the bond between the labelled peptides and the extracellular domain of IRAP was carried out by a binding test adapted from the test described by Demaegdt et al. (Demaegdt et al., 2004, Biochem. Pharmacol. 68, 885-892).

The extracellular domain of recombinant TRAP produced in affinity-purified insect cells (HighFive) was taken up in a 50 mM binding buffer Tris-HCl (pH 7.4) containing 140 mM of NaCl.

The incubations were carried out in 24-well plates in a final volume of 300 μL comprising 100 μL of the extracellular portion of TRAP, 50 μl of an EDTA/1,10-phenanthroline mixture with a final concentration of 500 μM of EDTA and 100 μM of 1,10-phenanthroline, and:
either 50 μl of binding buffer,
or 50 μl of binding buffer containing the unlabelled peptides for the competition tests.

50 μl of labelled peptides was added at a final concentration of 0.5 to 0.8 nM for the saturation tests, or at a concentration of 3 nM for the other tests.

The incubation was carried out at 37° C., at variable (kinetic) times up to 60 min.

The wells in which the extracellular portion of IRAP is adsorbed were then washed several times with incubation buffer in order to remove the free radioactivity.

The radioactivity was then measured using a scintillation counter, in the presence of scintillant on an ad hoc basis.

The affinity of the labelled peptides for the extracellular portion of IRAP was evaluated between 1 and 100 nM

### Example 5

### Stability of the Labelled Peptides

In order to measure the stability of the labelled peptides derived from angiotensin IV, their homologues labelled with cold iodine (¹²⁷I) were incubated at a concentration of 100 μM for 40 min in a serum sample, which serum was not treated beforehand with protease inhibitors.

The peptides were then separated from the serum by gel filtration and incubated at concentrations ranging from 0.1 nM to 1 μM with the purified extracellular domain of IRAP in the presence of [¹²⁵I]-AngIV (0.1-1 nM) or [¹²⁵I]-Nle-AngIV (0.1-1 nM) and peptidase and protease inhibitors.

The stability of the peptides in the serum sample is then measured indirectly by measuring the competition of the peptides labelled with ¹²⁵I with the bond of [¹²⁵I]-AngIV or [¹²⁵I]-Nle-AngIV to the extracellular domain of IRAP (cf. A. Lukaszuk et al. Med. Chem. 51, 2291-2296, 2008).

### Example 6

### IRAP RIA

### Principle of the Procedure

Assay of the circulating extracellular portion of IRAP (extIRAPc) according to the invention calls on to an immunoradiometric method. The circulating extracellular portion of IRAP present in samples originating from patients or standards is recognised by a monoclonal antibody (Mab) specific to the extracellular portion of TRAP, said monoclonal antibody being bound to the inner surface of polystyrene tubes.

The addition of a peptide labelled with Iodine 125 or with tritium (peptide*), having a high affinity for extIRAPc induces the formation of a sandwich complex bound to the solid phase (tube): Mab-extIRAPc-peptide*.

At the end of the incubation, the fraction of peptide* not bound to the solid phase is removed by aspiration and by washing. The formation of the complex only takes place in the presence of extIRAPc, the radioactivity bound to the solid phase (tube) is directly proportional to the concentration of extIRAPc in the sample. A standardization curve allows the concentration of extIRAPc in the samples to be assayed to be determined by interpolation.

### Sampling, Preparation and Storage of the Samples

The assay can be carried out on human serum. If the assay is carried out in the 24 hours following the sampling, the samples can be stored at 2-8° C. Otherwise, they must be aliquoted and frozen at −20° C. or at lower temperatures for a maximum of five months. If the samples have been frozen, they must be completely thawed and homogenized before the assay. Repeated freezing and thawing cycles must be avoided.

If high levels of extIRAPc are expected, it is necessary to dilute the sample with the zero standard (cf. below). The use of plastic tubes during the preparation of the dilutions is recommended.

### Assay Procedure Reconstitution of Lyophilized Reagents

Reconstitute the zero standard with 5 mL of distilled water, the zero standard corresponding to a sample having no extIRAPc.
Reconstitute the standards 1 to 6 (samples of serum for which the quantity of extIRAPc is known) and the controls G1 and G2 (G1 and G2 corresponding to solutions containing extIRAPc) with 1 mL of distilled water.

Reconstitute the reagents several minutes before use and mix gently (avoid the formation of foam).

DILUTION OF THE WASHING BUFFER: Add 900 mL of distilled water to 100 mL of concentrated washing buffer. Avoid the formation of foam. (washing buffer: PBS, albumin 0.1%, pH 7.4).

### Assay Protocol

Before use, the coated tubes, the standards, the control serums and the serums to be analyzed are placed at ambient temperature (18-25° C.) for at least 30 minutes; then their content is mixed with a Vortex. It is recommended that the analysis be carried out twice both for the standards, the control serums and the samples. Scrupulously keep to the order of use of the reagents:

1. Pipette 50 μL of each sample (standards, control serum and sample) and deposit at the bottom of as many tubes coated with Mab as there are samples taken, and in as many uncoated tubes as there are samples taken.
2. Distribute 200 μL of peptide* into each of the tubes including those that are uncoated intended for the evaluation of the total activity.
3. Incubate for 2 hours±5 minutes at ambient temperature (18-25° C.) with horizontal stirring (200-300 rpm).
4. Wash the tubes as follows: carefully aspirate the content of the tubes (except those intended for the evaluation of the total activity), add 1 mL of diluted washing buffer into each tube (except those intended for the evaluation of the total activity) and aspirate the washing solution. Repeat the washing twice. In order to obtain results which are reproducible and reliable it is necessary to carry out the washing correctly. Careful compliance with the incubation time and complete removal of the washing solution are fundamental for a successful analysis.
5. Measure the activity of all the tubes using a gamma counter, including those intended for the evaluation of the total activity.

### Quality Control

It is recommended that control samples be included in each analysis in order to verify the quality of the results obtained.

All the samples must be treated in the same way and the results are analysed with a suitable statistical method. If the values of the control serums are not included in the acceptability range indicated, the assay must be done again.

### Calculation of the Results

1. Calculate the average of the counts for each standard, control or sample.
2. Plot the standard curve using the average counts for each standard reported on the y-axis as a function of the respective concentrations of insulin on the x-axis.
3. Calculate the concentrations of the controls and the samples by interpolation of the standard curve using the respective average counts correcting, if appropriate, by the dilution factor used.

### Example of RIA

4G6: anti IRAP monoclonal antibody (IgG3a) directed against the peptide sequence QKKGKELFIQQER (peptide sequence of IRAP No 3)
Tube: BSA-biotin-avidin-4G6-biot
IRAP-HIS recombinant protein (lot712-CAP-05p) at 410 μg/ml
   Range between 0 and 12.5 μg/ml
“IRAP-HIS” buffer range: PBS (11 mM PO4+140mM Nacl)+1% BSA free protease+0.1% proclin+5 mM EDTA
Conditioning Buffer tracers=Buffer IRAP-HIS+120-M Phenanthroline
Tracers, fractions F1 of the ligands labelled at 1 μci/ml
Ligand 6
Ligand 11
Ligand 17
Angiotensin IV
Washing solution: H2O+0.05% Tween 20

### Protocol

200 μl/tube IRAP-His
   Incubation 4 h AT under stirring (750 rpm)
2 washes=aspiration+(2×2 ml/tube)
200 μl ligand*/tube
   Incubation overnight at AT
2 washes=aspiration+(2×2 ml/tube)
counting the radioactivity over 1 mn

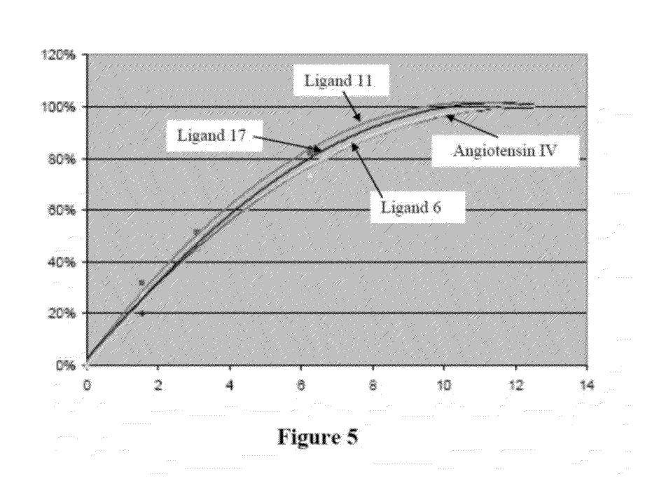

The results are presented in FIG. 5.

1. Method for the assay of the circulating extracellular portion of the IRAP protein (“insulin responsive aminopeptidase”) comprising at least one stage of quantitative assay of said purified, secreted, extracellular portion of IRAP, by means of at least one modified and labelled peptide,
said labelled peptide interacting specifically with said extracellular portion of IRAP,
said peptide being modified by the presence of at least one non-natural amino acid, and/or at least one natural or non-natural β-homo amino acid,
said labelled peptide being preferentially labelled with at least one radioactive isotope,
provided that
said peptide is not an antibody and
said modified peptide, labelled with an iodine ¹²⁵I atom, is different from the sequence Nle-Tyr-Ile-His-Pro-Phe (SEQ ID NO 23).

2. Method for the assay of the circulating extracellular portion of IRAP according to claim 1, where said modified and labelled peptide is a ligand of IRAP which is
either a labelled substrate of the IRAP protein, chosen from
labelled and modified [Arg]-vasopressin,
labelled and modified Oxytocin,
labelled and modified Met-/Leu-enkephalin,
labelled and modified Somatostatin,
labelled and modified CCK-8,
labelled and modified Neurokinin A,
labelled and modified Neuromedin B,
labelled and modified Lys-bradykinin, and
labelled and modified Dynorphin A,
or a labelled peptide inhibiting the IRAP protein, chosen from
labelled and modified Angiotensin IV, and
labelled and modified LVV-hemorphin-7.

3. Method for the assay of the circulating extracellular portion of IRAP according to claim 1, comprising at least:
a stage of purification of said circulating extracellular portion of IRAP, and
a stage of quantification of said circulating extracellular portion purified in the previous stage, by means of at least one labelled modified peptide as previously defined.

4. Method for the assay of the circulating extracellular portion of IRAP according to claim 1, where said purification of the circulating portion of the IRAP protein is carried out by means of at least one antibody specific to said extracellular portion of IRAP, in particular by immunoprecipitation or immunocapture.

5. Method for the assay of the circulating extracellular portion of IRAP according to claim 1, where said IRAP protein is represented by
the IRAP protein of sequence SEQ ID NO 1, or
a protein homologous to the IRAP protein exhibiting at least 80% sequence identity with the sequence SEQ ID NO 1, provided that said homologous protein is capable of interacting specifically with the IRAP ligands or the IRAP inhibiting peptides, or
an isoform of the IRAP protein, said isoform being the product of the alternative splicing of the RNA encoding the IRAP protein SEQ ID NO 1, provided that said isoform is capable of interacting specifically with the IRAP ligands or the IRAP inhibiting peptides, said isoforms of the IRAP protein SEQ ID NO 1 being in particular represented by the proteins SEQ ID NO 2 or 3.

6. Method of assay according to claim 5, where
said variants of the IRAP protein possess an amino acid sequence chosen from the sequences SEQ ID NO 4 to SEQ ID NO 7, and
said isoforms of the IRAP protein possess an amino acid sequence chosen from the sequences SEQ ID NO 8 to SEQ ID NO 15.

7. Method of assay according to claim 1, in which said circulating extracellular portion of the IRAP protein possesses an amino acid sequence represented by the sequences chosen from the following sequences: SEQ ID NO 16, SEQ ID NO 17, SEQ ID NO 18, SEQ ID NO 19 and SEQ ID NO 20.

8. Method of assay according to claim 1, where said labelled modified peptide is labelled with at least one tritium atom or at least one atom of iodine ¹²⁵I, preferentially a single atom of iodine ¹²⁵I.

9. Method of assay according to claim 1, where said labelled modified peptide is represented by general formula (III) below: 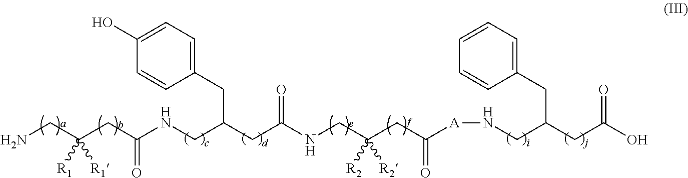
where a, b, c, d, e, f, g, h, i and j can be equal to 0 or 1, so that (a+b)≦1, (c+d)≦1, (e+f)≦1, (g+h)≦1 and (i,j)≦1, (a,b), (c,d), (e,f), (g,h) and (i,j) being independent of each other,
where (R₁, R₁′) are such that:
R1 is chosen from: a —CH(CH₃)₂ group, a —CH₂—CH(CH₃)₂ group, a —CH(CH₃)—CH₂—CH₃ group, a —(CH₂)₃—CH₃ group and a C(CH₃)₃ group, and R1′ is a hydrogen atom, or
R1 and R1′ form together with the carbon which carries them a cyclopentyl,
where (R₂, R₂′) are such that:
if R2 is chosen from: a —CH₂—CH(CH₃)₂ group, a —CH(CH₃)—CH₂—CH₃ group, a —(CH₂)₃—CH₃ group and a C(CH₃)₃ group, R2′ is a hydrogen atom, or
R2 and R2′ form together with the carbon which carries them a cyclopentyl,
the pairs (R₁, R₁′) and (R₂, R₂′) being chosen independently of each other,
where A is chosen from the following groups 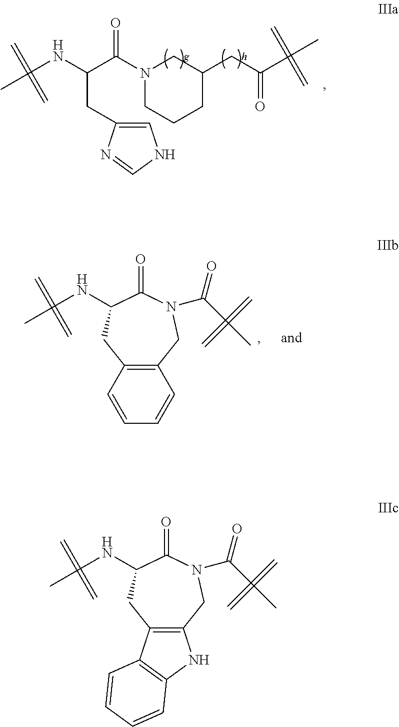
said labelled modified peptide being in the form of a racemate, any one of its enantiomers, or any one of the different tautomers corresponding to said racemates and enantiomers,
provided that if A is represented by formula IIIa and if a, b, c, d, e, f, g, h, i and j are equal to 0,
if (R₁, R₁′)═(—CH(CH₃)₂;H) then (R₂, R₂′) is different from (—CH(CH₃)—CH₂—CH₃;H), and
if (R₂, R₂′)═(—CH(CH₃)—CH₂—CH₃;H) then (R₁, R₁′) is different from (—CH(CH₃)₂;H)
and provided that if said modified peptide is labelled with an iodine ¹²⁵I atom, if a, b, c, d, e, f, g, h, i and j are equal to 0 then (R₁, R₁′) is different from (—(CH₂)₃—CH₃;H).

10. Method of assay according to claim 1, where said labelled and modified peptide consists of the following sequence:

| | |
|---|---|
| X1-X2-X3-His-X4-X5 | (SEQ ID NO 22) |

where
X1 can be a Valine (Val), a Leucine (Leu), an Isoleucine (Ile), a Norleucine (Nle), a Cycloleucine (Cle) or a tert-leucine (Tle), or a β² or β³ derivative of one of these amino acids,
X2 can be a tyrosine, or a β² or β³ derivative of tyrosine,
X3 can be a Leucine (Leu), an Isoleucine (Ile), a Norleucine (Nle), a Cycloleucine (Cle) or a tert-leucine (Tle), or a β² or β³ derivative of one of these amino acids,
X4 can be a proline or a β² or β³ derivative of proline,
X5 can be a phenylalanine, or a β² or β³ derivative of phenylalanine,
and (His-X4) which can be
an aba-gly of formula IIa 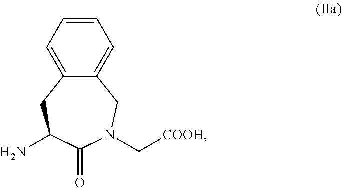
or
an aia-gly of formula IIb 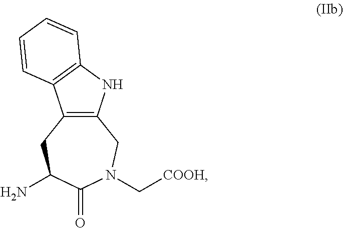
at least one of the amino acids X1 to X5 is a non-natural amino acid, and/or a natural or non-natural β-homo amino acid, provided that the sequence SEQ ID NO 22 labelled with an iodine ¹²⁵I atom is different from the sequence Nle-Tyr-Ile-His-Pro-Phe (SEQ ID NO 23).

11. Method of assay according to claim 9, where said labelled and modified peptide consists of the following sequence:

| | |
|---|---|
| X1-X2-X3-His-X4-X5 | (SEQ ID NO 22) |

and where at least one of the amino acids X1 to X5 is a natural or non-natural β-homo amino acid.

12. Method of assay according to claim 9, where said labelled modified peptide is labelled
with at least one iodine ¹²⁵I atom on the tyrosine X2, preferentially a single iodine ¹²⁵I atom, in particular on the phenyl group, or
by the replacement of at least one of the hydrogen atoms with a tritium ³H atom.

13. Method of assay according to claim 9, where said labelled modified peptide is chosen from the following peptides:

| | |
|---|---|
| β²hVal-Tyr-Ile-His-Pro-Phe-, | (SEQ ID NO 24) |
| β²hVal-Tyr-Ile-His-Pro-β³hPhe-, | (SEQ ID NO 25) |
| β²hLeu-Tyr-Ile-His-Pro-β³hPhe-, | (SEQ ID NO 26) |
| Val-Tyr-Ile-His-β²hPro-Phe-, | (SEQ ID NO 27) |
| β²hVal-Tyr-Ile-Aba-Gly-Phe-, | (SEQ ID NO 28) |
| and | |
| β²hVal-Tyr-Ile-Aia-Gly-Phe-, | (SEQ ID NO 29) |

preferentially the peptide SEQ ID NO 25,
said labelled modified peptide being iodinated, preferentially by a single iodine ¹²⁵I atom.

14. Method of assay according to claim 9, where said labelled modified peptide is chosen from the following peptides:

| | |
|---|---|
| β2hVal-Tyr-Ile-His-Pro-Phe-, | (SEQ ID NO 24) |
| β2hVal-Tyr-Ile-His-Pro-β3hPhe-, | (SEQ ID NO 25) |
| β2hLeu-Tyr-Ile-His-Pro-β3hPhe-, | (SEQ ID NO 26) |
| Val-Tyr-Ile-His-β²hPro-Phe-, | (SEQ ID NO 27) |
| β²hVal-Tyr-Ile-Aba-Gly-Phe-, | (SEQ ID NO 28) |
| and | |
| β²hVal-Tyr-Ile-Aia-Gly-Phe-, | (SEQ ID NO 29) |

preferentially the peptide SEQ ID NO 25,
said labelled modified peptide being tritiated.

15. Method of assay according to claim 1, comprising
a stage of purification of said circulating extracellular portion of IRAP, and
a stage of quantification of said circulating extracellular portion purified in the previous stage, by means of at least one modified peptide of amino acid sequence SEQ ID NO 25 labelled with radioactive iodine ¹²⁵I, preferentially with a single iodine ¹²⁵I atom.

16. Method of assay according to claim 1, comprising
a stage of purification of said circulating extracellular portion of IRAP, and
a stage of quantification of said circulating extracellular portion purified in the previous stage, by means of at least one tritiated labelled modified peptide of amino acid sequence SEQ ID NO 25.

17. Modified labelled peptide represented by general formula (III) below: 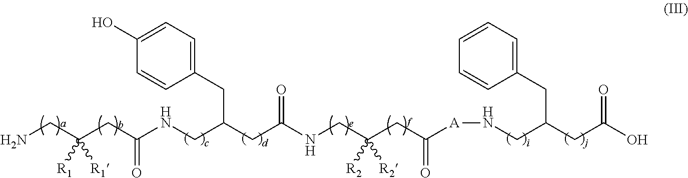
where a, b, c, d, e, f, g, h, i and j can be equal to 0 or 1, so that (a+b)≦1, (c+d)≦1, (e+f)≦1, (g+h)≦1 and (i,j)≦1, (a,b), (c,d), (e,f), (g,h) and (i,j) being independent of each other,
where (R₁, R₁′) are such that:
R1 is chosen from: a —CH(CH₃)₂ group, a —CH₂—CH(CH₃)₂ group, a —CH(CH₃)—CH₂—CH₃ group, a —(CH₂)₃—CH₃ group and a C(CH₃)₃ group, and R1′ is a hydrogen atom, or
R1 and R1′ form together with the carbon which carries them a cyclopentyl,
where (R₂, R₂′) are such that:
if R2 is chosen from: a —CH₂—CH(CH₃)₂ group, a —CH(CH₃)—CH₂—CH₃ group, a —(CH₂)₃—CH₃ group and a C(CH₃)₃ group, R2′ is a hydrogen atom, or
R2 and R2′ form together with the carbon which carries them a cyclopentyl,
the pairs (R₁, R₁′) and (R₂, R₂′) being chosen independently of each other,
where A is chosen from the following groups 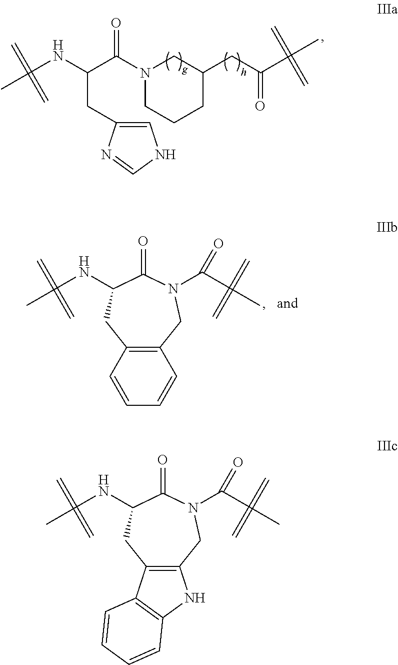
said modified labelled peptide being in the form of a racemate, any one of its enantiomers, or any one of the different tautomers corresponding to said racemates and enantiomers,
provided that if A is represented by formula IIIa and if a, b, c, d, e, f, g, h, i and j are equal to 0,
if (R₁, R₁′)═(—CH(CH₃)₂;H) then (R₂, R₂′) is different from (—CH(CH₃)—CH₂—CH₃;H), and
if (R₂, R₂′)═(—CH(CH₃)—CH₂—CH₃;H) then (R₁, R₁′) is different from (—CH(CH₃)₂;H)
and provided that if said modified peptide is labelled with an iodine ¹²⁵I atom, if a, b, c, d, e, f, g, h, i and j are equal to 0 then (R₁, R₁′) is different from (—(CH₂)₃—CH₃;H).

18. Modified labelled peptide, said peptide being labelled with at least one radioactive isotope consisting of the following sequence:

| | |
|---|---|
| X1-X2-X3-His-X4-X5, | (SEQ ID NO 22) |

said peptide being modified,
where
X1 can be a Valine (Val), a Leucine (Leu), an Isoleucine (Ile), or a non-natural amino acid derived from leucine, in particular Norleucine (Nle), Cycloleucine (Cle) or tert-leucine (Tle), or a β² or β² derivative of one of these natural or non-natural amino acids,
X2 can be a tyrosine, or a β² or β³ derivative of tyrosine,
X3 can be a Leucine (Leu), an Isoleucine (Ile), or a non-natural amino acid derived from leucine, in particular Norleucine (Nle), Cycloleucine (Cle) or tert-leucine (Tle), or a β² or β² derivative of one of these natural or non-natural amino acids,
X4 can be proline or a β² or β² derivative of proline,
X5 can be a phenylalanine, or a β² or β² derivative of phenylalanine,
and (His-X4) which can be
an aba-gly of formula IIa 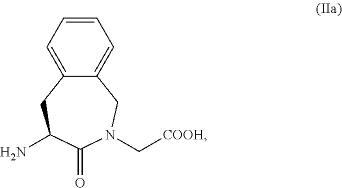
or
an aia-gly of formula IIb 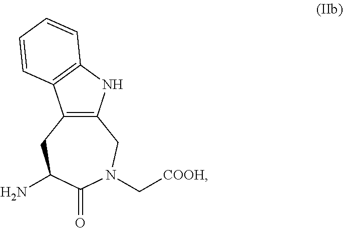
and
provided that said labelled unmodified peptide is different from angiotensin IV, and in particular that said labelled unmodified peptide is different from the sequence SEQ ID NO 21 (Val-Tyr-Ile-His-Pro-Phe),
provided that the sequence SEQ ID NO 22 labelled with an iodine ¹²⁵I atom is different from the sequence Nle-Tyr-Ile-His-Pro-Phe (SEQ ID NO 23).

19. Labelled modified peptide according to claim 18, consisting of the following sequence:

| | |
|---|---|
| X1-X2-X3-His-X4-X5, | (SEQ ID NO 22) |

said peptide being modified so that at least one of the amino acids X1 to X5 is a natural or non-natural β-homo amino acid.

20. Labelled modified peptide according to claim 18, said labelled modified peptide being labelled
by at least one iodine ¹²⁵I atom on the tyrosine X2, preferentially a single iodine ¹²⁵I atom, in particular on the phenyl group, or
by the replacement of at least one of the hydrogen atoms with a tritium ³H atom.

21. Labelled modified peptide according to claim 18, where said labelled modified peptide is chosen from the following peptides:

| | |
|---|---|
| β2hVal-Tyr-Ile-His-Pro-Phe-, | (SEQ ID NO 24) |
| β2hVal-Tyr-Ile-His-Pro-β3hPhe-, | (SEQ ID NO 25) |
| β2hLeu-Tyr-Ile-His-Pro-β3hPhe-, | (SEQ ID NO 26) |
| Val-Tyr-Ile-His-β²hPro-Phe-, | (SEQ ID NO 27) |
| β²hVal-Tyr-Ile-Aba-Gly-Phe-, | (SEQ ID NO 28) |
| and | |
| β²hVal-Tyr-Ile-Aia-Gly-Phe-, | (SEQ ID NO 29) |

preferentially the peptide SEQ ID NO 25,
said labelled modified peptide being iodinated, preferentially by a single iodine ¹²⁵I atom.

22. Labelled modified peptide according to claim 18, where said labelled modified peptide is chosen from the following peptides:

| | |
|---|---|
| β2hVal-Tyr-Ile-His-Pro-Phe-, | (SEQ ID NO 24) |
| β2hVal-Tyr-Ile-His-Pro-β3hPhe-, | (SEQ ID NO 25) |
| β2hLeu-Tyr-Ile-His-Pro-β3hPhe-, | (SEQ ID NO 26) |
| Val-Tyr-Ile-His-β²hPro-Phe-, | (SEQ ID NO 27) |
| β²hVal-Tyr-Ile-Aba-Gly-Phe-, | (SEQ ID NO 28) |
| and | |
| β²hVal-Tyr-Ile-Aia-Gly-Phe-, | (SEQ ID NO 29) |

preferentially the peptide SEQ ID NO 25,
said labelled modified peptide being tritiated.
